(19) United States
(12) Patent Application Publication
(10) Pub. No.: US 2023/0285606 A1
(43) Pub. Date: Sep. 14, 2023

(54) METHODS AND AGENTS FOR THE DETECTION AND TREATMENT OF CANCER

(71) Applicant: CASE WESTERN RESERVE UNIVERSITY, Cleveland, OH (US)

(72) Inventor: SUSANN BRADY-KALNAY, Wellington, OH (US)

(21) Appl. No.: 18/019,993

(22) PCT Filed: Oct. 6, 2021
(86) PCT No.: PCT/US21/53867

§ 371 Date: Feb. 6, 2023

Related U.S. Application Data
(60) Provisional application No. 63/062,053, filed on Aug. 6, 2020.

(51) Int. Cl. A61K 51/08; G01N 33/574; A61K 47/64; A61K 49/00; A61P 35/00

(52) U.S. Cl. CPC A61K 51/08; G01N 33/57484; A61K 47/64; A61K 49/0002; A61P 35/00

(57) ABSTRACT

An agent for use in detecting, monitoring, and/or imaging cancer cells and/or cancer cell metastasis, migration, dispersal, and/or invasion, and/or for treating cancer in a subject includes a targeting peptide, at least one of a detectable moiety, therapeutic agent, or a theranostic agent, and a peptide or peptidomimetic spacer that directly or indirectly links the targeting peptide to the at least one of the detectable moiety, therapeutic agent, or theranostic agent.

### RELATED APPLICATION

This application claims priority of U.S. Provisional Application No. 63/062,053 filed Aug. 6, 2020, the subject matter of which is incorporated herein by reference in its entirety.

### BACKGROUND

Cancer detection and treatment are hindered by the inability to differentiate between cancer cells and normal cells. Better detection tools for cancer or tumor imaging are needed for earlier diagnosis of cancers. Molecular recognition of tumor cells would facilitate guided surgical resection. In order to improve surgical resection, targeted imaging tools must specifically label tumor cells, not only in the main tumor but also along the edge of the tumor and in the small tumor cell clusters that disperse throughout the body.

Targeted imaging tools designed to label molecules that accumulate in the tumor microenvironment may also be advantageous as therapeutic targeting agents, as they can identify both the main tumor cell population and areas with infiltrating cells that contribute to tumor recurrence. The ability to directly target the tumor cell and/or its microenvironment would increase both the specificity and sensitivity of current treatments, therefore reducing non-specific side effects of chemotherapeutics that affect cells throughout the body.

### SUMMARY

Embodiments described herein relate to an agent as well as its use in detecting, monitoring, and/or imaging cancer cells and/or cancer cell metastasis, migration, dispersal, and/or invasion, and/or for treating cancer in a subject. The agent can include a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment; at least one of a detectable moiety, therapeutic agent, or theranostic agent; and a peptide or peptidomimetic spacer that directly or indirectly links the targeting peptide to the at least one of the detectable moiety, therapeutic agent, or theranostic agent. The peptide or peptidomimetic spacer has a length and structure effective to at least maintain or preserve binding affinity of the linked targeting peptide to the proteolytically cleaved extracellular fragment and activity of the at least one of the linked detectable moiety, therapeutic agent, or a theranostic agent.

In some embodiments, the agent is configured for in vivo administration to a subject or ex vivo administration to a biological sample of the subject.

In some embodiments, the spacer includes natural and/or non-natural amino acids.

In other embodiments, the spacer includes at least 3 natural or non-natural amino acids. For example, the spacer can have a length of 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids.

In some embodiments, the spacer includes at least about 50%, at least about 60%, at least about 70%, at least about 80%, or at least about 90% glycine and/or serine residues.

In other embodiments, the spacer includes at least 50%, at least 60%, at least 70%, or at least 80% glycine residues.

In some embodiments, the spacer is a polyglycine or glycine/serine spacer.

In some embodiments, the spacer includes the amino acid sequence of at least one of (GS)a, (GGS)b, or (GGGS)c, or (GGGGS)d and wherein a, b, c, and d are each independently 2, 3, 4, 5, or 6. For example, the spacer can have an amino acid sequence of

(SEQ ID NO: 9)
GGG,
(SEQ ID NO: 10)
GGGG,
(SEQ ID NO: 11)
GGGGG,
(SEQ ID NO: 12)
GGGGGG,
(SEQ ID NO: 13)
GGGGGGG,
(SEQ ID NO: 14)
GGGGGGGG,
(SEQ ID NO: 15)
GGGGGGGGG,
(SEQ ID NO: 16)
GSGS,
(SEQ ID NO: 17)
GSGSGS,
(SEQ ID NO: 18)
GSGSGSGS,
(SEQ ID NO: 19)
GSGSGSGSGS,
(SEQ ID NO: 20)
GGSGGS,
(SEQ ID NO: 21)
GGSGGSGGS,
(SEQ ID NO: 22)
GGSGGSGGSGGS,
(SEQ ID NO: 23)
GGGSGGGS,
(SEQ ID NO: 24)
GGGSGGGSGGGS,
(SEQ ID NO: 25)
GGGSGGGSGGGSGGGS,
(SEQ ID NO: 26)
GGGGSGGGGS,
or
(SEQ ID NO: 27)
GGGGSGGGGSGGGGS.

In some embodiments, the agent further includes at least one coupling agent that links the spacer to the targeting peptide and/or the at the at least one of the detectable moiety, therapeutic agent, or theranostic agent.

In some embodiments, the cell adhesion molecule can include a cell surface receptor protein tyrosine phosphatase (PTP) type IIb.

In some embodiments, the extracellular fragment can include the amino acid sequence of SEQ ID NO: 2, and the targeting peptide can include a polypeptide that specifically binds to and/or complexes to SEQ ID NO: 2.

In some embodiments, the targeting peptide can include a polypeptide having an amino acid sequence that has at least 80% sequence identity to about 10 to about 50 consecutive amino acids of SEQ ID NO: 3.

In other embodiments, the targeting peptide can include a polypeptide having an amino acid sequence selected from the group consisting of SEQ ID NO: 4, SEQ ID NO: 5, SEQ ID NO: 6, SEQ ID NO: 7, and SEQ ID NO: 8.

In some embodiments, the detectable moiety can include a chelating agent, contrast agent, imaging agent, radiolabel, semiconductor particle, nanoparticle, nanobubble, or nanochain. The detectable moiety can be detectable by at least one of magnetic resonance imaging (MRI), positron emission tomography (PET) imaging, computer tomography (CT) imaging, gamma imaging, near infrared imaging, ultrasound or fluorescent imaging.

In some embodiments, the theranostic or therapeutic agent includes at least one of a photosensitizer, ultrasound sensitizer, thermal sensitizer, radiosenstizer, radiotherapeutic, chemotherapeutic, or immunotherapeutic.

In some embodiments, the cancer detected or treated with the agent can be of any type of cancer including, but not limited to, bone cancer, bladder cancer, brain cancer, neuroblastoma, breast cancer, cancer of the urinary tract, carcinoma, cervical cancer, astrocytoma, brain stem glioma, glioblastoma, neuroendocrine tumors, NCS atypical teratoid/rhabdoid tumor, CNS embryonal tumor, CNS Germ Cell tumors, craniopharyngioma, ependymoma, kidney tumors, acute lymphoblastic leukemia, acute myeloid leukemia, and other types of leukemia; Hodgkin lymphoma, non-Hodgkin lymphoma, Ewing sarcoma, osteosarcoma and malignant fibrous histiocytoma of the bone, rhabdomyosarcoma, soft tissue sarcoma, Wilms' tumor, colon cancer, esophageal cancer, gastric cancer, head and neck cancer, hepatocellular cancer, liver cancer, lung cancer, lymphoma and leukemia, melanoma, ovarian cancer, endometrial cancer, pancreatic cancer, pituitary cancer, prostate cancer, rectal cancer, renal cancer, sarcoma, stomach cancer, testicular cancer, thyroid cancer, and uterine cancer.

In some embodiments, the cancer cell can be, for example, a metastatic, migrating, dispersed, and/or invasive cancer cell, such as a metastatic, migrating, dispersed, and/or invasive brain cancer cell (e.g., glioma cell and, specifically, a glioblastoma multiforme (GBM) cell), lung cancer cell, breast cancer cell, prostate cancer cell, ovarian cancer, endometrial cancer cell, and/or melanoma.

Other embodiments described herein relate to a method of detecting cancer cells and/or cancer cell metastasis, migration, dispersal, and/or invasion in a subject in need thereof. The method includes administering to the subject an amount of an agent described herein wherein the agent includes a diagnostic agent or a theranostic agent. The agent bound to and/or complexed with the cancer cells can be detected to determine the location and/or distribution of the cancer cells in the subject.

In some embodiments, the cancer cells include at least one of a glioma, lung cancer, melanoma, breast cancer, ovarian cancer cell, endometrial cancer cell, or prostate cancer cell.

In some embodiments, the agent can be administered systemically to the subject.

In some embodiments, the agent can be detected to define a tumor margin in a subject.

Other embodiments described herein relate to a method of treating cancer in a subject in need thereof. The method includes administering to the subject a therapeutically effective amount of an agent as described herein that includes a therapeutic or theranostic agent.

In some embodiments, the therapeutic agent or theranostic agent is a photosensitizer, radiosensitizer, or radiotherapeutic, and the method can further include irradiating cancer cells to which the agent is bound or internalized by, thereby inducing the photosensitizing or radiosensitizing effects of the photosensitizer or radiotherapeutic and apoptosis and/or necrosis of the cancer cells. The photosensitizer can include, for example, a porphyrin, tricarbocyanine, or pthalocyanine compound.

In other embodiments, the therapeutic agent or theranostic agent is a nanobubble, and the method can further include insonating nanobubbles bound or internalized by to cancer cells with ultrasound energy effective to promote inertial cavitation and apoptosis and/or necrosis of the cancer cell and/or release a chemotherapeutic to the cancer cells.

### BRIEF DESCRIPTION OF THE DRAWINGS

FIG. 1 illustrates plots showing in vivo average radiant efficiency of a first agent without a peptide spacer, a second agent having a peptide spacer, and control agent having a peptide spacer administered to mice with heterotopic xenograft flank U87 tumor implants.

FIG. 2 illustrates plots showing the in vivo average radiant efficiency of the second agent having the peptide spacer and a third agent having a different peptide spacer administered to mice with heterotopic xenograft U87 flank tumor implants or mice with heterotopic xenograft flank U87 tumor implants overexpressing PTPmu.

FIG. 3 illustrates plots showing the in vivo average radiant efficiency of the third agent having a peptide spacer and a fourth agent having a different peptide spacer administered to mice with heterotopic xenograft U87 flank tumor implants or mice with heterotopic xenograft flank U87 tumor implants overexpressing PTPmu.

FIG. 4 illustrates ex vivo images and a graph showing average radiant efficiency of the first agent and a control agent following in vivo administration to mice with heterotopic xenograft U87 flank tumor implants.

FIG. 5 illustrates ex vivo images and graphs showing average radiant efficiency of the second agent, the third agent, and control agents following in vivo administration to mice with heterotopic xenograft U87 flank tumor implants.

FIG. 6 illustrates ex vivo images and a graph showing average radiant efficiency of the second agent, the third agent, and control agents following in vivo administration to mice with heterotopic xenograft U87 flank tumor implants overexpressing PTPmu.

FIG. 7 illustrates ex vivo images and a graph showing average radiant efficiency of the third agent, the fourth agent, and control agents following in vivo administration to mice with heterotopic xenograft U87 flank tumor implants overexpressing PTPmu.

FIG. 8 illustrates ex vivo images and a graph showing average radiant efficiency of the first agent compared to a control agent following in vivo administration to mice with orthotopic xenograft U87 intracranial tumors

FIG. 9 illustrates ex vivo images and a graph showing average radiant efficiency of the third agent compared to a control agent following in vivo administration to mice with orthotopic xenograft U87 intracranial tumors.

FIG. 10 illustrates ex vivo images of orthotopic xenograft U87 intracranial tumors or orthotopic xenograft LN229 intracranial tumors following in vivo administration to mice of the third agent, the fourth agent, and control agents.

FIG. 11 illustrates ex vivo maestro images overlaid on black and white photographs of the brain following in vivo administration to mice of the third agent, the fourth agent, and control agents.

FIG. 12 illustrates a graph showing maximum signal intensity of the third agent, the fourth agent, and control agents following in vivo administration to mice with orthotopic xenograft U87 intracranial tumors.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, uterus, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiforme, meningoma, medulloblastoma, schwannoma and epidymoma.

The term “chimeric protein” or “fusion protein” is a fusion of a first amino acid sequence encoding a polypeptide with a second amino acid sequence defining a domain (e.g., polypeptide portion) foreign to and not substantially homologous with any domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

The term “epitope” includes any protein determinant capable of specific binding to an immunoglobulin. Epitope determinants usually consist of chemically active surface groupings of molecules such as amino acids or sugar side chains and usually have specific three-dimensional structural characteristics, as well as specific charge characteristics.

The term “gene” or “recombinant gene” refers to a nucleic acid comprising an open reading frame encoding a polypeptide, including both exon and (optionally) intron sequences.

The term “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “peptide(s)”, “protein(s)” and “polypeptide(s)” are used interchangeably herein. As used herein, “polypeptide” refers to any peptide or protein comprising two or more amino acids joined to each other by peptide bonds or modified peptide bonds (i.e., peptide isomers). “Polypeptide(s)” refers to both short chains, commonly referred as peptides, oligopeptides or oligomers, and to longer chains generally referred to as proteins.

The terms “polynucleotide sequence” and “nucleotide sequence” are also used interchangeably herein.

“Recombinant,” as used herein, means that a protein is derived from a prokaryotic or eukaryotic expression system.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “wild type” refers to the naturally-occurring polynucleotide sequence encoding a protein, or a portion thereof, or protein sequence, or portion thereof, respectively, as it normally exists in vivo.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to agents for use in detecting, monitoring, and/or imaging cancer cells and/or cancer cell metastasis, migration, dispersal, and/or invasion in a subject, methods of detecting, monitoring, and/or imaging cancer cells and/or cancer cell metastasis, migration, dispersal, and/or invasion in a subject, methods of determining and/or monitoring the efficacy of a cancer therapeutic and/or cancer therapy administered to a subject in need thereof, and methods of treating a cancer in a subject in need thereof using the agents.

The agents described herein include a targeting peptide that specifically binds to and/or complexes with a proteolytically cleaved extracellular fragment of an immunoglobulin (Ig) superfamily cell adhesion molecule in the cancer cell microenvironment that is expressed by the cancer cell or an endothelial cell, which supports survival of the cancer cell, at least one of a detectable moiety, therapeutic agent, or a theranostic agent, and a peptide or peptidomimetic spacer that directly or indirectly links the targeting peptide to the at least one of the detectable moiety, therapeutic agent, or theranostic agent.

It was found that a peptide or peptidomimetic spacer that directly or indirectly links the targeting peptide to the at least one of the detectable moiety, therapeutic agent, or theranostic agent can be selected such that the peptide or peptidomimetic spacer has a length and structure effective to at least maintain, preserve, or not interfere with binding affinity of the linked targeting peptide to the proteolytically cleaved extracellular fragment and activity of the at least one of the linked detectable moiety, therapeutic agent, or theranostic agent. By activity of the detectable moiety or theranostic agent it is meant, for example, the ability of the detectable moiety or theranostic agent to be detected or imaged in vivo, ex vivo, or in vitro by magnetic resonance imaging (MRI), positron emission tomography (PET) imaging, computer tomography (CT) imaging, gamma imaging, near infrared imaging, ultrasound imaging, fluorescent imaging, or other detection means. By activity of the therapeutic agent or the theranostic agent it is meant, for example, the biological, physiological, or pharmacological activity of the therapeutic agent or theranostic agent to treat a disease or condition (e.g., treating cancer).

For example, when the agent includes a detectable moiety that is directly or indirectly linked by the peptide or peptidomimetic spacer to the targeting peptide, the agent was found to clearly demarcate tumor cells in tissue sections and tumor “edge” samples, suggesting that the agent can be used as a diagnostic tool for molecular imaging of metastatic, dispersive, migrating, or invading cancers or the tumor margin. Systemic introduction of agent as described herein resulted in rapid and specific labeling of the flank tumors and intracranial tumors within minutes. Labeling occurred primarily within the tumor, however a gradient of agent at the tumor margin was also observed. There is also a signal amplification effect as extracellular fragments accumulate over time.

The agents can be administered systemically to a subject and readily target cancer cells associated with proteolytically cleaved extracellular fragment of the immunoglobulin (Ig) superfamily cell adhesion molecule, such as metastatic, migrating, dispersed, and/or invasive cancer cells. In some embodiments, the agent after systemic administration can cross the blood brain barrier to define cancer cell location, distribution, metastases, dispersions, migrations, and/or invasion as well as tumor cell margins in the subject. In other embodiments, the agent after systemic administration can inhibit and/or reduce cancer cell survival, proliferation, and migration.

The agents described herein can therefore be used in a method of detecting cancer cells and/or cancer cell metastasis, migration, dispersal, and/or invasion as well as in a method of treating cancer in a subject in need thereof. The methods can include administering to a subject an agent that includes a targeting peptide that binds to and/or complexes with the proteolytically cleaved extracellular fragment of the Ig superfamily cell adhesion molecule in the cancer cell or tumor cell microenvironment, at least one detectable moiety, and a peptide or peptidomimetic spacer that directly or indirectly links the targeting peptide to the at least one of the detectable moiety. The agent bound to and/or complexed with the cancer cells can be detected to determine the location and/or distribution of the cancer cells in the subject.

In some embodiments, the Ig superfamily cell adhesion molecule can include an extracellular homophilic binding portion, which can bind in homophilic fashion or engage in homophilic binding in a subject. In one example, the Ig superfamily cell adhesion molecule includes RPTP type IIb cell adhesion molecules. In another example, Ig superfamily cell adhesion molecules can include RPTPs of the PTPμ-like subfamily, such as PTPμ, PTPκ, PTPρ, and PCP-2 (also called PTPλ). PTPμ-like RPTPs include a MAM (Meprin/A5-protein/PTPμ) domain, an Ig domain, and FNIII repeats. PTPμcan have the amino acid sequence of SEQ ID NO: 1, which is identified by Genbank Accession No. AAI51843.1. It will be appreciated that the PTPμgene can generate splice variants such that the amino acid sequence of PTPμcan differ from SEQ ID NO: 1. In some embodiments, PTPμcan have an amino acid sequence identified by Genbank Accession No. AAH51651.1 and Genbank Accession No. AAH40543.1.

Cancer cells and/or endothelial cells, which support cancer cell survival, that express an Ig superfamily cell adhesion molecule and that can be proteolytically cleaved to produce a detectable extracellular fragment can include, for example, cancer cells and/or other cells in the tumor microenvironment, such as stem cells, endothelial cells, stromal cells and immune cells that promote their survival.

The cancers detected and/or treated by the agents described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or ureter); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The agents can also be used to detect and/or treat a variety of cancers or other abnormal proliferative diseases, including (but not limited to) the following: carcinoma, including that of the bladder, breast, prostate, rectal, colon, kidney, liver, lung, ovary, uterus, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoictic tumors of myeloid lineage, including acute and chronic myelogenous leukemias and promyclocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, glioblastoma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are detected, treated or prevented in the skin, lung, colon, rectum, breast, prostate, bladder, kidney, pancreas, ovary, or uterus. In other specific embodiments, sarcoma, melanoma, or leukemia is detected and/or treated.

In still other embodiments, the cancer cells that are detected and/or treated can include glioma cells, lung cancer cells, breast cancer cells, prostate cancer cells, and melanoma cells, such as invasive, dispersive, motile or metastatic cancer cells can include glioma cells, lung cancer cells, breast cancer cells, prostate cancer cells, and melanoma cells. It will be appreciated that other cancer cells and/or endothelial cells, which support cancer cell survival, that express an Ig superfamily cell adhesion molecule and that can be proteolytically cleaved to produce a detectable extracellular fragment can identified or determined by, for example, using immunoassays that detect the Ig superfamily cell adhesion molecule expressed by the cancer cells or endothelial cells.

In some embodiments, the targeting peptide (or targeting polypeptide) can include a polypeptide (or targeting polypeptide) that binds to and/or complexes with the proteolytically cleaved extracellular fragment of the Ig superfamily cell adhesion molecule. The targeting peptide can include, consist essentially of, or consist of about 10 to about 50 amino acids and have an amino acid sequence that is substantially homologous or identical to about 10 to about 50 consecutive amino acids of a homophilic binding portion or domain of the proteolytically cleaved extracellular fragment of the Ig superfamily cell adhesion molecule. By substantially homologous, it is meant the targeting polypeptide has an amino acid sequence at least about 80%, about 90%, about 95%, about 96%, about 97%, about 98%, about 99% or about 100% identical to a portion of the amino acid sequence of the binding portion of the proteolytically cleaved extracellular fragment of the Ig superfamily cell adhesion molecule.

In one example, the homophilic binding portion of the Ig superfamily cell adhesion molecule can include, for example, the Ig domain of the cell adhesion molecule. In another example, where the Ig superfamily cell adhesion molecule is PTPμ, the homophilic binding portion can include the Ig binding domain and the MAM domain.

In another aspect, the targeting peptide can have an amino acid sequence that is substantially homologous to about 10 to about 50 consecutive amino acids of the Ig binding domain and/or MAM domain of PTPμ(e.g., SEQ ID NO: 1) and readily cross the blood brain barrier when systemically administered to a subject. The development of the PTPμtargeting peptides can be based on a large body of structural and functional data. The sites required for PTPμ-mediated homophilic adhesion have been well characterized. In addition, the crystal structure of PTPμcan provide information regarding which regions of each functional domain are likely to be exposed to the outside environment and therefore available for homophilic binding and thus detection by a peptide.

In some embodiments, the proteolytically cleaved extracellular fragment of PTPμ(e.g., SEQ ID NO: 1) can include an amino acid sequence of SEQ ID NO: 2, the Ig and MAM binding region can comprise the amino acid sequence of SEQ ID NO: 3, and the polypeptide can have an amino acid sequence that is substantially homologous to about 10 to about 50 consecutive amino acids of SEQ ID NO: 2 or SEQ ID NO: 3. Examples of polypeptides that can specifically bind SEQ ID NO: 2 or SEQ ID NO: 3 and have an amino acid sequence that is substantially homologous to about 10 to about 50 consecutive amino acids of SEQ ID NO: 2 or SEQ ID NO: 3 are polypeptides that include an amino acid sequence selected from the group consisting of SEQ ID NO: 4, SEQ ID NO: 5 (SBK2), SEQ ID NO: 6, and SEQ ID NO: 7. Polypeptides comprising SEQ ID NO: 4, 5, 6, or 7 can recognize or bind to the MAM, Ig domain, or the FNIII repeats. In particular embodiments, the targeting peptide is a SBK2 polypeptide comprising the amino acid sequence SEQ ID NO:5.

In other embodiments, a polypeptide that binds to and/or complexes with the proteolytically cleaved extracellular fragment of the Ig superfamily CAM or its receptor that is expressed by a cancer cell or another cell in the cancer cell microenvironment can have the amino acid sequence of SEQ ID NO: 8. SEQ ID NO: 8 is substantially homologous to a portion of SEQ ID NO: 1 or SEQ ID NO: 2 and can specifically bind to SEQ ID NO: 2 or SEQ ID NO: 3.

The targeting peptides can be subject to various changes, substitutions, insertions, and deletions where such changes provide for certain advantages in its use. In this regard, targeting peptides that bind to and/or complex with a proteolytically cleaved extracellular portion of an Ig superfamily cell adhesion molecule can be substantially homologous with, rather than be identical to, the sequence of a recited polypeptide where one or more changes are made and it retains the ability to function as specifically binding to and/or complexing with the proteolytically cleaved extracellular portion of an Ig superfamily cell adhesion molecule.

The targeting peptides can be in any of a variety of forms of polypeptide derivatives, that include amides, conjugates with proteins, cyclized polypeptides, polymerized polypeptides, retro-inverso peptides, analogs, fragments, chemically modified polypeptides, and the like derivatives.

The term “analog” includes any polypeptide having an amino acid residue sequence substantially identical to a sequence specifically shown herein in which one or more residues have been conservatively substituted with a functionally similar residue and that specifically binds to and/or complexes with the proteolytically cleaved extracellular portion of an Ig superfamily CAM as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein also include any polypeptide having one or more additions and/or deletions or residues relative to the sequence of a polypeptide whose sequence is shown herein, so long as the requisite activity is maintained.

Retro-inverso peptides are linear peptides whose amino acid sequence is reversed and the α-center chirality of the amino acid subunits is inverted as well. These types of peptides are designed by including D-amino acids in the reverse sequence to help maintain side chain topology similar to that of the original L-amino acid peptide and make them more resistant to proteolytic degradation. D-amino acids represent conformational mirror images of natural L-amino acids occurring in natural proteins present in biological systems. Peptides that contain D-amino acids have advantages over peptides that just contain L-amino acids. In general, these types of peptides are less susceptible to proteolytic degradation and have a longer effective time when used as pharmaceuticals. Furthermore, the insertion of D-amino acids in selected sequence regions as sequence blocks containing only D-amino acids or in-between L-amino acids allows the design of peptide based drugs that are bioactive and possess increased bioavailability in addition to being resistant to proteolysis. Furthermore, if properly designed, retro-inverso peptides can have binding characteristics similar to L-peptides.

The term “fragment” refers to any subject polypeptide having an amino acid residue sequence shorter than that of a polypeptide whose amino acid residue sequence is shown herein.

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine and the like) and optionally substituted ethanolamines (e.g., ethanolamine, diethanolamine and the like).

The targeting peptides can be synthesized by any of the techniques that are known to those skilled in the peptide art, including recombinant DNA techniques. Synthetic chemistry techniques, such as a solid-phase Merrifield-type synthesis, can be used for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

It will be appreciated that the targeting peptide can bind to and/or complex with homophilic binding domains of proteolytically cleaved extracellular fragments of other Ig superfamily cell adhesion molecules, besides PTPs. For example, a similar molecular detection strategy described herein can be used with any other Ig superfamily CAM having a homophilic binding cell surface protein whose ligand binding site is known. A large variety of cell surface proteins, including other phosphatases, are cleaved at the cell surface (Streuli M, Saito H (1992) Expression of the receptor-linked protein tyrosine phosphatase LAR: proteolytic cleavage and shedding of the CAM-like extracellular region. EMBO J 11:897-907; Anders L, Ullrich A (2006) Furin-, ADAM 10-, and gamma-secretase-mediated cleavage of a receptor tyrosine phosphatase and regulation of beta-catenin's transcriptional activity. Mol Cell Biol 26:3917-3934; Haapasalo A, Kovacs D M (2007) Presenilin/gamma-secretase-mediated cleavage regulates association of leukocyte-common antigen-related (LAR) receptor tyrosine phosphatase with beta-catenin. J Biol Chem 282:9063-9072; Chow J P, Noda M (2008) Plasmin-mediated processing of protein tyrosine phosphatase receptor type Z in the mouse brain. Neurosci Lett 442:208-212; Craig S E, Brady-Kalnay S M. Tumor-derived extracellular fragments of receptor protein tyrosine phosphatases (RPTPs) as cancer molecular diagnostic tools. Anticancer Agents Med Chem. 2011 January; 11(1):133-40. Review. PubMed PMID: 21235433; PubMed Central PMCID: PMC3337336; Craig S E, Brady-Kalnay S M. Cancer cells cut homophilic cell adhesion molecules and run. Cancer Res. 2011 Jan. 15; 71(2):303-9. Epub 2010 Nov. 17. PubMed PMID: 21084269; PubMed Central PMCID: PMC3343737; Phillips-Mason P J, Craig S E, Brady-Kalnay S M. Should I stay or should I go? Shedding of RPTPs in cancer cells switches signals from stabilizing cell-cell adhesion to driving cell migration. Cell Adh Migr. 2011 Jul. 1; 5(4):298-305. Epub 2011 Jul. 1. PubMed PMID: 21785275; PubMed Central PMCID: PMC3210297). These proteins represent additional targets for that can be readily used by the skilled artisan for forming therapeutic polypeptides that can be used to treat cancers (Barr A J, Ugochukwu E, Lee W H, King O N, Filippakopoulos P, Alfano I, Savitsky P, Burgess-Brown N A, Muller S, Knapp S (2009) Large-scale structural analysis of the classical human protein tyrosine phosphatome. Cell 136:352-363).

In some embodiments, the targeting peptides described herein can include additional residues that may be added at either terminus of a polypeptide for the purpose of providing a “linker” by which the polypeptides can be conveniently linked and/or affixed to the peptide or peptidomimetic spacer. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

The peptide or peptidomimetic spacer that directly or indirectly links the targeting peptide to the at least one of the detectable moiety, therapeutic agent, or theranostic agent can include additional natural and/or non-natural amino acid residues added at either terminus of a targeting peptide (or target peptide with linker peptide). The peptide or peptidomimetic spacer can include at least three natural or non-natural amino acids and have a structure effective to at least maintain or preserve binding affinity of the linked targeting peptide to the proteolytically cleaved extracellular fragment and activity of the at least one of the linked detectable moiety, therapeutic agent, or a theranostic agent. Typical amino acid residues used for use in the spacer are glycine, serine tyrosine, cysteine, lysine, glutamic and aspartic acid, or the like.

In some embodiments, the peptide or peptidomimetic spacer is selected in part based on its ability to alter the phobicity (e.g., to cause the agent to become more hydrophilic or hydrophobic) depending on its desired use.

In some embodiments, the spacer can be a flexible peptide or peptidomimetic spacer that directly or indirectly links the targeting peptide to other polypeptides, proteins, and/or molecules, such as detectable moieties, labels, therapeutic agents, theranostic agents, solid matrices, or carriers. A flexible peptide or peptidomimetic spacer can be, for example, at least about 3 to about 30 or fewer natural or non-natural amino acids in length. For example, the spacer can have a length of 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids. Where the spacer is a peptide spacer, the peptide spacer may be produced as a single recombinant polypeptide using a conventional molecular biological/recombinant DNA method.

In some embodiments, the spacer includes at least about 50%, at least about 60%, at least about 70%, at least about 80%, or at least about 90% glycine and/or serine residues.

In other embodiments, the spacer includes at least 50%, at least 60%, at least 70%, or at least 80% glycine residues. In some embodiments, the balance of the spacer includes serine residues.

In some embodiments, the spacer is a polyglycine or glycine/serine spacer that consists of purely glycine residues or glycine and serine residues. The small size of glycine residues provides flexibility and allows mobility of the connecting targeting peptide and at least one of detectable moiety, therapeutic agent, or theranostic agent. The incorporation of serine can maintain the stability of the spacer in aqueous solutions by forming hydrogen bonds with water molecules and therefore can reduce unfavorable interactions between the spacer and targeting peptide.

In some embodiments, the spacer includes the amino acid sequence of at least one of (GS)a, (GGS)b, or (GGGS)c, or (GGGGS)d and wherein a, b, c, and d are each independently 2, 3, 4, 5, or 6. For example, the spacer can have an amino acid sequence of

(SEQ ID NO: 9)
GGG,
(SEQ ID NO: 10)
GGGG,
(SEQ ID NO: 11)
GGGGG,
(SEQ ID NO: 12)
GGGGGG,
(SEQ ID NO: 13)
GGGGGGG,
(SEQ ID NO: 14)
GGGGGGGG,
(SEQ ID NO: 15)
GGGGGGGGG,
(SEQ ID NO: 16)
GSGS,
(SEQ ID NO: 17)
GSGSGS,
(SEQ ID NO: 18)
GSGSGSGS,
(SEQ ID NO: 19)
GSGSGSGSGS,
(SEQ ID NO: 20)
GGSGGS,
(SEQ ID NO: 21)
GGSGGSGGS,
(SEQ ID NO: 22)
GGSGGSGGSGGS,
(SEQ ID NO: 23)
GGGSGGGS,
(SEQ ID NO: 24)
GGGSGGGSGGGS,
(SEQ ID NO: 25)
GGGSGGGSGGGSGGGS,
(SEQ ID NO: 26)
GGGGSGGGGS,
or
(SEQ ID NO: 27)
GGGGSGGGGSGGGGS.

In some embodiments, the spacer can be a contiguous portion of the targeting peptide that is coupled directly to an N terminus or C terminus residue of the targeting peptide with or without a linker peptide.

For example, a polyglycine or glycine/serine space coupled to a SBK2 targeting peptide having SEQ ID NO: 5 can have the amino acid sequence of:

(SEQ ID NO: 28)
GGG.GEGDDFNWEQVNTLTKPTSD,
(SEQ ID NO: 29)
GGGG.GEGDDFNWEQVNTLTKPTSD,
(SEQ ID NO: 30)
GGGGG.GEGDDFNWEQVNTLTKPTSD,
(SEQ ID NO: 31)
GGGGGG.GEGDDFNWEQVNTLTKPTSD,
(SEQ ID NO: 32)
GGGGGGG.GEGDDFNWEQVNTLTKPTSD,
(SEQ ID NO: 33)
GGGGGGGG.GEGDDFNWEQVNTLTKPTSD,
(SEQ ID NO: 34)
GGGGGGGGG.GEGDDFNWEQVNTLTKPTSD,
(SEQ ID NO: 35)
GSGS.GEGDDFNWEQVNTLTKPTSD,
(SEQ ID NO: 36)
GSGSGS.GEGDDFNWEQVNTLTKPTSD,
(SEQ ID NO: 37)
GSGSGSGS.GEGDDFNWEQVNTLTKPTSD,
(SEQ ID NO: 38)
GSGSGSGSGS.GEGDDFNWEQVNTLTKPTSD,
(SEQ ID NO: 39)
GGSGGS.GEGDDFNWEQVNTLTKPTSD,
(SEQ ID NO: 40)
GGSGGSGGS.GEGDDFNWEQVNTLTKPTSD,
(SEQ ID NO: 41)
GGSGGSGGSGGS.GEGDDFNWEQVNTLTKPTSD,
(SEQ ID NO: 42)
GGGSGGGS.GEGDDFNWEQVNTLTKPTSD,
(SEQ ID NO: 43)
GGGSGGGSGGGS.GEGDDFNWEQVNTLTKPTSD,
(SEQ ID NO: 44)
GGGSGGGSGGGSGGGS.GEGDDFNWEQVNTLTKPTSD,
(SEQ ID NO: 45)
GGGGSGGGGS.GEGDDFNWEQVNTLTKPTSD,
or
(SEQ ID NO: 46)
GGGGSGGGGSGGGGS.GEGDDFNWEQVNTLTKPTSD.

It will be appreciated that other peptide or peptidomimetics spacer can be linked to SBK2 or other targeting peptides described herein at the N terminus or C terminus portion of the targeting peptide.

In some embodiments, targeting peptides with a contiguous spacer can be produced as a recombinant polypeptide. For the production of recombinant polypeptides, a variety of host organisms may be used. Examples of hosts include, but are not limited to: bacteria, such as E. coli, yeast cells, insect cells, plant cells and mammalian cells. The skilled artisan will understand how to take into consideration certain criteria in selecting a suitable host for producing the recombinant polypeptide. Factors affecting selection of a host include, for example, post-translational modifications, such as phosphorylation and glycosylation patterns, as well as technical factors, such as the general expected yield and the ease of purification. Host-specific post-translational modifications of the targeting peptide or spacer peptide, which is to be used in vivo, should be carefully considered because certain post-translational modifications are known to be highly immunogenic.

In other embodiments, the spacer can be non-contiguous portion of the targeting peptide that is indirectly coupled or conjugated to the targeting peptide via coupling agent or conjugating agent. By a “non-contiguous portion” it is meant that the targeting peptide and spacer are connected via an additional element that is not a part and/or peptide residue of the targeting peptide or spacer that is contiguous in nature and functions as a linker.

The coupling agent and/or conjugating agent can include, for example, maleimidyl binders, which can be used to bind to thiol groups, isothiocyanate and succinimidyl (e.g., N-hydroxysuccinimidyl (NHS)) binders, which can bind to free amine groups, diazonium which can be used to bind to phenol, and amines, which can be used to bind with free acids such as carboxylate groups using carbodiimide activation. Useful functional groups can be present on the peptide or peptidomimetic spacer based on the particular amino acids present, and additional groups can be designed. It will be evident to those skilled in the art that a variety of bifunctional or polyfunctional reagents, both homo- and hetero-functional (such as those described in the catalog of the Pierce Chemical Co., Rockford, Ill.), can be employed as a coupling agent. Coupling can be effected, for example, through amino groups, carboxyl groups, sulfhydryl groups or oxidized carbohydrate residues.

Examples of coupling agent and/or conjugating agent are described in Means and Feeney, CHEMICAL MODIFICATION OF PROTEINS, Holden-Day, 1974, pp. 39-43. Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other coupling agents or conjugating agents include: p,p′-difluoro-m,m′-dinitrodiphenylsulfone (which forms irreversible linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

The coupling agent or conjugating may be homobifunctional, i.e., having two functional groups that undergo the same reaction. An example of a homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible linking of polypeptides that contain cysteine residues.

Coupling agents or conjugating agents may also be heterobifunctional. Heterobifunctional coupling or conjugating agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (“SMCC”), m-maleimidobenzoyl-N-hydroxysuccinimide ester (“MBS”), and succinimide 4-(p-maleimidophenyl) butyrate (“SMPB”), an extended chain analog of MBS. The succinimidyl group of these cross-linkers reacts with a primary amine, and the thiol-reactive maleimide forms a covalent bond with the thiol of a cysteine residue.

Many coupling agents or conjugating agents yield a conjugate that is essentially non-cleavable under cellular conditions. However, some agents contain a covalent bond, such as a disulfide, that is cleavable under cellular conditions. For example, Traut's reagent, dithiobis (succinimidylpropionate) (“DSP”), and N-succinimidyl 3-(2-pyridyldithio) propionate (“SPDP”) are well-known cleavable cross-linkers. The use of a cleavable coupling or conjugating agents permits separation of the targeting peptide, spacer, and/or detectable moiety, therapeutic agent, and/or theranostic agent after delivery to the target cell. Direct disulfide linkage may also be useful.

Numerous coupling agents, including the ones discussed above, are commercially available. Detailed instructions for their use are readily available from the commercial suppliers. A general reference on protein cross-linking and conjugate preparation is: Wong, CHEMISTRY OF PROTEIN CONJUGATION AND CROSS-LINKING, CRC Press (1991).

In some embodiments, the peptide or peptidomimetic spacer can be directly or indirectly coupled to a detectable moiety, therapeutic, and/or theranostic agent, using for example a coupling agent or conjugating agent described herein.

In some embodiments, the detectable moiety can include any contrast agent or detectable label that facilitate the detection step of a diagnostic or therapeutic method by allowing visualization of the complex formed by binding of the agent comprising the targeting peptide, spacer, and detectable moiety and/or theranostic agent to the proteolytically cleaved extracellular fragment of the Ig superfamily cell adhesion molecule. The detectable moiety can be selected such that it generates a signal, which can be measured and whose intensity is related (preferably proportional) to the amount of the agent bound to the tissue being analyzed. Methods for labeling biological molecules, such as polypeptides are well-known in the art.

Any of a wide variety of detectable moieties can be linked with the targeting peptide by the peptide or peptidomimetic spacer described herein. Examples of detectable moieties include, but are not limited to: various ligands, radionuclides, fluorescent agents and dyes, infrared and near infrared agents, chemiluminescent agents, microparticles or nanoparticles (e.g., quantum dots, nanocrystals, semiconductor particles, nanoparticles, nanobubbles, or nanochains and the like), enzymes (e.g., those used in an ELISA, i.e., horseradish peroxidase, beta-galactosidase, luciferase, alkaline phosphatase), colorimetric labels, magnetic labels, chelating agents, biotin, dioxigenin or other haptens and proteins for which antisera or monoclonal antibodies are available.

In some embodiments, the agents including the detectable moiety described herein may be used in conjunction with non-invasive imaging (e.g., neuroimaging) techniques for in vivo imaging of the agent, such as magnetic resonance spectroscopy (MRS) or imaging (MRI), or gamma imaging, such as positron emission tomography (PET) or single-photon emission computed tomography (SPECT). The term “in vivo imaging” refers to any method, which permits the detection of a labeled agent, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of agent along with a large excess of unlabeled, but otherwise chemically identical compound.

For purposes of in vivo imaging, the type of detection instrument available is a major factor in selecting a given detectable moiety. For instance, the type of instrument used will guide the selection of the stable isotope. The half-life should be long enough so that it is still detectable at the time of maximum uptake by the target, but short enough so that the host does not sustain deleterious effects.

In one example, the detectable moiety can include a radiolabel, that is directly or indirectly linked (e.g., attached or complexed) with the peptide or peptidomimetic spacer using general organic chemistry techniques. The radiolabel can, for example, ⁶⁸Ga, ¹²³I, ¹³¹I, ¹²⁵I, ¹⁸F, ¹¹C, ⁷⁵Br, ⁷⁶Br, ¹²⁴I, ¹³N, ⁶⁴Cu, ³²P, ³⁵S. Such radiolabels can be detected by PET techniques, such as described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, N Y 1986) the contents of which are hereby incorporated by reference. The detectable moiety can also include ¹²³I for SPECT. The ¹²³I can be coupled to the peptide spacer by any of several techniques known to the art. See, e.g., Kulkarni, Int. J. Rad. Appl. & Inst. (Part B) 18: 647 (1991), the contents of which are hereby incorporated by reference. In addition, detectable moiety can include any radioactive iodine isotope, such as, but not limited to ¹³¹I, ¹²⁵I, or ¹²³I. The radioactive iodine isotopes can be coupled to the peptide spacer by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art.

The detectable moiety can further include known metal radiolabels, such as Technetium-99m (^{99m}Tc), ¹⁵³Gd, ¹¹¹In, ⁶⁷Ga, ²⁰¹Tl, ⁸²Rb, ⁶⁴Cu, ⁹⁰Y ¹⁸⁸Rh, T (tritium), ¹⁵³Sm, ⁸⁹Sr, and ²¹¹At. Modification of the targeting peptide to introduce ligands that bind such metal ions can be effected without undue experimentation by one of ordinary skill in the radiolabeling art. The metal radiolabeled agents can then be used to detect cancers, such as GBM in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

In some embodiments, the detectable moiety can include a chelating agent (with or without a chelated radiolabel metal group). Examples chelating agents can include those disclosed in U.S. Pat. No. 7,351,401, which is herein incorporated by reference in its entirety. In some embodiments, the chelating agent is 1,4,7,10-tetraazacyclododecane-1,4,7,10-tetraacetic acid (DOTA).

Fluorescent labeling agents or infrared agents include those known to the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, ALEXA 647, IR700, IR800, INDOCYANINE GREEN (ICG), TEXAS RED, or QUASAR 670.

Fluorescent labeling agents can also include other known fluorophores, or proteins known to the art, for example, green fluorescent protein. The disclosed targeting peptides and peptide or peptidomimetic spacer can be directly or indirectly coupled to the fluorescent labeling agents, administered to a subject or a sample, and the subject/sample examined by fluorescence spectroscopy or imaging to detect the labeled compound.

In some embodiments, the detectable moiety includes a fluorescent dye. Exemplary Fluorescent dyes include fluorescein isothiocyanate, cyanines, such as Cy5, Cy5.5 and analogs thereof (e.g., sulfo-Cyanine 5 NHS ester and Cy5.5 maleimide). See also Handbook of Fluorescent Probes and Research Chemicals, 6th Ed., Agents, Inc., Eugene Oreg., which is incorporated herein by reference.

The detectable moiety can further include a near infrared imaging group. Near infrared imaging groups are disclosed in, for example, Tetrahedron Letters 49(2008) 3395-3399; Angew. Chem. Int. Ed. 2007, 46, 8998-9001; Anal. Chem. 2000, 72, 5907; Nature Biotechnology vol 23, 577-583; Eur Radiol (2003) 13: 195-208; and Cancer 67: 1991 2529-2537, which are herein incorporated by reference in their entirety. Applications may include the use of a NIRF (near infra-red) imaging scanner. In one example, the NIRF scanner may be handheld. In another example, the NIRF scanner may be miniaturized and embedded in an apparatus (e.g., micro-machines, scalpel, neurosurgical cell removal device).

Quantum dots, e.g., semiconductor particles, can also be employed as detectable moieties as described in Gao, et al “In vivo cancer targeting and imaging with semiconductor quantum dots”, Nature Biotechnology, 22, (8), 2004, 969-976, the entire teachings of which are incorporated herein by reference. The disclosed targeting peptides and peptide or peptidomimetic spacer can be coupled to the quantum dots, administered to a subject or a sample, and the subject/sample examined by fluorescence spectroscopy or imaging to detect the labeled compound.

In certain embodiments, a detectable moiety includes an MRI contrast agent. MRI relies upon changes in magnetic dipoles to perform detailed anatomic imaging and functional studies. MRI can employ dynamic quantitative T1 mapping as an imaging method to measure the longitudinal relaxation time, the T1 relaxation time, of protons in a magnetic field after excitation by a radiofrequency pulse. T1 relaxation times can in turn be used to calculate the concentration of an agent in a region of interest, thereby allowing the retention or clearance of an agent to be quantified. In this context, retention is a measure of molecular contrast agent binding.

Numerous magnetic resonance imaging (MRI) contrast agents are known to the art, for example, positive contrast agents and negative contrast agents. The disclosed targeting peptides and peptide or peptidomimetic spacer can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iron oxide, or the like. Typical contrast agents include macrocycle-structured gadolinium(III)chelates, such as gadoterate meglumine (gadoteric acid), gadopentetate dimeglumine, gadoteridol, mangafodipir trisodium, gadodiamide, and others known to the art. In certain embodiments, the detectable moiety includes gadoterate meglumine Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

In some embodiments, the targeting peptide and peptide or peptidomimetic spacer can can be coupled or linked to a chelating agent, such as macrocyclic chelator DOTA, and a single metal radiolabel.

In other embodiments, the targeting peptide and peptide or peptidomimetic spacer or a plurality of the targeting peptides and peptide or peptidomimetic spacers can be coupled or linked to a nanobubble for diagnostic and/or therapeutic applications. The nanobubble can include a lipid membrane that defines internal void that includes at least one gas. Examples of nanobubbles that can be coupled to the targeting peptide and peptide or peptidomimetic spacer are described, for example, in U.S. Pat. Nos. 10,375,575, 10,434,194, and 10,973,935 as well as U.S. Patent Application Publication Nos. 2029/0061220 and 2021/0106699, all of which are incorporated by reference in their entirety.

The agent comprising the detectable moiety described herein can be administered to the subject by, for example, systemic, topical, and/or parenteral methods of administration. These methods include, e.g., injection, infusion, deposition, implantation, or topical administration, or any other method of administration where access to the tissue by the agent is desired. In one example, administration of the agent can be by intravenous injection of the agent in the subject. Single or multiple administrations of the probe can be given. “Administered”, as used herein, means provision or delivery of the agent in an amount(s) and for a period of time(s) effective to label cancer cells in the subject.

Agents described herein that include a detectable moiety can be administered to a subject in a detectable quantity of a pharmaceutical composition containing an agent or a pharmaceutically acceptable water-soluble salt thereof, to a patient.

Formulation of the agent to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A “detectable quantity” means that the amount of the detectable compound that is administered is sufficient to enable detection of binding of the compound to the cancer cells. An “imaging effective quantity” means that the amount of the detectable compound that is administered is sufficient to enable imaging of binding of the agent to the cancer cells.

The agents including the detectable moiety administered to a subject can be used in a method to detect and/or determine the presence, location, and/or distribution of cancer cells, i.e., cancer cells associated with proteolytically cleaved extracellular fragments of Ig superfamily cell adhesion molecules, in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the agent in the animal's tissue, e.g., brain tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the agent may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the composition. In this case, the baseline and post-administration images can be compared to ascertain the presence, absence, and/or extent of a particular disease or condition.

In one aspect, the agent including the detectable moiety may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination. For example, in glioma (brain tumor) surgery, the agents can be given intravenously about 24 hours prior to pre-surgical stereotactic localization MRI. The agents can be imaged on gradient echo MRI sequences as a contrast agent that localizes with the glioma.

Agents described herein that include a detectable moiety and specifically bind to and/or complex with proteolytically cleaved Ig superfamily cell adhesion molecules (PTPμ) associated with cells can be used in intra-operative imaging (IOI) techniques to guide surgical resection and eliminate the “educated guess” of the location of the tumor margin by the surgeon. Previous studies have determined that more extensive surgical resection improves patient survival Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Fluorescence-guided resection of glioblastoma multiforme by using 5-aminolevulinic acid-induced porphyrins: a prospective study in 52 consecutive patients. Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Thus, agents that function as diagnostic molecular imaging agents have the potential to increase patient survival rates.

In some embodiments, to identify and facilitate removal of cancers cells, microscopic intra-operative imaging (III) techniques can be combined with systemically administered or locally administered agents described herein. The agents upon administration to the subject can target and detect and/or determine the presence, location, and/or distribution of cancer cells, i.e., cancer cells associated with proteolytically cleaved extracellular fragments of Ig superfamily cell adhesion molecules, in an organ or body area of a patient. In one example, the agent can be combined with IOI to identify malignant cells that have infiltrated and/or are beginning to infiltrate at a tumor brain margin. The method can be performed in real-time during brain or other surgery. The method can include local or systemic application of the targeted agent described herein that includes a detectable moiety, e.g., a fluorescent or MRI contrast moiety. An imaging modality can then be used to detect and subsequently gather image data. The imaging modality can include one or combination of known imaging techniques capable of visualizing the agent. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

In one example, an agent including a targeting peptide and peptide or peptidomimetic spacer linked to a fluorescent detectable moiety can be topically applied as needed during surgery to interactively guide a surgeon and/or surgical instrument to remaining abnormal cells. The agent may be applied locally in low concentration, making it unlikely that pharmacologically relevant concentrations are reached. In one example, excess material may be removed (e.g., washed off) after a period of time (e.g., incubation period).

Another embodiment described herein relates to a method of monitoring the efficacy of a cancer therapeutic or cancer therapy administered to a subject. The methods and agents described herein can be used to monitor and/or compare the invasion, migration, dispersal, and metastases of a cancer in a subject prior to administration of a cancer therapeutic or cancer therapy, during administration, or post therapeutic regimen.

A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The cancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA.

The method of monitoring the efficacy of a cancer therapeutic can include the steps of administering in vivo to the animal an agent as described herein, then visualizing a distribution of the agent in the animal (e.g., with an in vivo imaging modality as described herein), and then correlating the distribution of the agent with the efficacy of the cancer therapeutic. It is contemplated that the administering step can occur before, during, and after the course of a therapeutic regimen in order to determine the efficacy of a chosen therapeutic regimen. One way to assess the efficacy of the cancer therapeutic is to compare the distribution of an agent pre and post cancer therapy.

In some embodiments, the agent bound to and/or complexed with the proteolytically cleaved extracellular fragment of the Ig superfamily cell adhesion molecule is detected in the subject to detect and/or provide the location and/or distribution of the cancer cells in the subject. The location and/or distribution of the cancer cells in the subject can then be compared to a control to determine the efficacy of the cancer therapeutic and/or cancer therapy. The control can be the location and/or distribution of the cancer cells in the subject prior to the administration of the cancer therapeutic and/or cancer therapy. The location and/or distribution of the cancer cells in the subject prior to the administration of the cancer therapeutic and/or cancer therapy can be determined by administering the agent to the subject and detecting the agent bound to and/or complexed with cancer cells in the subject prior to administration of the cancer therapeutic and/or cancer therapy.

In certain embodiments, the methods and agents described herein can be used to measure the efficacy of a therapeutic administered to a subject for treating a metastatic, invasive, or dispersed cancer. In this embodiment, the agent can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the agent can be used to define the distribution of the metastatic cancer pre-operative and post-operative to determine the efficacy of the surgical resection. Optionally, the methods and agents can be used in an intra-operative surgical procedure as describe above, such as a surgical tumor resection, to more readily define and/or image the cancer cell mass or volume during the surgery.

In other embodiments, the targeting peptide and peptide or peptidomimetic spacer can be directly or indirectly linked to a therapeutic agent or theranostic agent. In one example, the theranostic or therapeutic agent linked to the targeting peptide and peptide or peptidomimetic spacer can used in a method of treating cancer or tumors (e.g., brain cancer or tumors). In one embodiment, the therapeutic agent or theranostic agent can include a photosensitizer and an agent comprising the targeting peptide, spacer, and photosensitizer can be used in photodynamic therapy.

Photodynamic therapy (PDT) is a site specific treatment modality that requires the presence of a photosensitizer, light, and adequate amounts of molecular oxygen to destroy targeted tumors (Grossweiner, Li, The science of phototherapy. Springer: The Netherlands, 2005). Upon illumination, a photoactivated sensitizer transfers energy to molecular oxygen that leads to the generation of singlet oxygen (O₂) and other reactive oxygen species (ROS), which initiate apoptosis and oxidative damage to cancer cells. Only the cells that are exposed simultaneously to the theranostic PDT drug (which is non-toxic in the dark) and light are destroyed while surrounding healthy, non-targeted and nonirradiated cells are spared from photodamage. Furthermore, the fluorescence of the photosensitizer molecules enables simultaneous diagnostic optical imaging that can be used to guide the PDT cancer treatment.

Methods for conducting photodynamic therapy are known in the art. See for example Thierry Patrice. Photodynamic Therapy; Royal Society of Chemistry, 2004. A pharmaceutical composition including an agent, which comprises a targeting peptide, a spacer, and a theranostic agent directly or indirectly linked to the spacer, can be applied to an organ or tissue as a step in PDT. In certain embodiments, the composition is applied to an epithelial, mesothelial, synovial, fascial, or serosal surface, including, but not limited to, the eye, esophagus, mucous membrane, bladder, joint, tendon, ligament, bursa, gastrointestinal, genitourinary, pleural, pericardial, pulmonary, or uroepithelial surfaces.

A theranostic agent or therapeutic agent for PDT directly or indirectly linked to the spacer and targeting peptide can be administered to a subject with cancer by systemic administration, such as intravenous administration. Upon administration, the targeted agent can localize to and/or accumulate at the site of the targeted tumor or cancer. In some embodiments, specific binding and/or complexing with a proteolytically cleaved extracellular fragment of an immunoglobulin (Ig) superfamily cell adhesion molecule that is expressed by a cancer cell or another cell in the cancer cell microenvironment allows the agent including the targeting peptide, spacer, and PDT agent to be bound to, complexed with and/or taken up by the targeted cells by, for example, endocytosis. This binding and/or uptake is specific to the targeted cells, which allows selective targeting of the cancer cells and/or cells in the cancer cell microenvironment in the subject by the targeted agents.

Following administration and localization of an agent, which includes the targeting peptide, spacer, and theranostic agent or therapeutic agent, to the targeted cancer cells, the targeted cancer cells can be exposed to therapeutic amount of light that causes cancer cell damage and/or suppression of cancer cell growth. The light, which is capable of activating the PDT agent can delivered to the targeted cancer cells using, using for example, semiconductor laser, dye laser, optical parametric oscillator or the like. It will be appreciated that any source light can be used as long as the light excites the hydrophobic PDT agent.

By way of example, agents including a targeting peptide, spacer, and a PDT agent can provide image guidance for glioma tumor resection and allow for subsequent PDT to eliminate unresectable or remaining cancer cells. In certain embodiments, the targeting moiety can comprise a peptide having SEQ ID NO:5.

PDT agent photosensitizer compounds for use in an agent described herein can include compounds that are excited by an appropriate light source to produce radicals and/or reactive oxygen species. Typically, when a sufficient amount of photosensitizer appears in diseased tissue (e.g., tumor tissue), the photosensitizer can be activated by exposure to light for a specified period. The light dose supplies sufficient energy to stimulate the photosensitizer, but not enough to damage neighboring healthy tissue. The radicals or reactive oxygen produced following photosensitizer excitation kill the target cells (e.g., cancer cells). The light treatment of tissue where PDT agents accumulate can also induce an immune response. In some embodiments, the targeted tissue can be locally illuminated. For example, light can be delivered to a photosensitizer via an argon or copper pumped dye laser coupled to an optical fiber, a double laser consisting of KTP (potassium titanyl phosphate)/YAG (yttrium aluminum garnet) medium, LED (light emitting diode), or a solid state laser.

PDT sensitizers for use as a theranostic or therapeutic agent can include a first generation photosensitizer (e.g., hematoporphyrin derivatives (HpDs) such as Photofrin (porfimer sodium), Photogem, Photosan-3 and the like). In some embodiments, PDT sensitizers can include second and third generation photosensitizers such as porphyrinoid derivatives and precursors. Porphyrinoid derivatives and precursors can include porphyrins and mettaloporphrins (e.g., meta-tetra(hydroxyphenyl)porphyrin (m-THPP), 5,10,15,20-tetrakis(4-sulfanatophenyl)-21H,23H-porphyrin (TPPS4), and precursors to endogenous protoporphyrin IX (PpIX): 5-aminolevulinic acid (5-ALA, which has been used for photodynamic therapy (PDT) of gliomas with some success (Stummer, W. et al. J Neurooncol. 2008, 87(1):103-9), methyl aminolevulinate (MAL), hexaminolevulinate (HAL)), chlorins (e.g., benzoporphyrin derivative monoacid ring A (BPD-MA), meta-tetra(hydroxyphenyl)chlorin (m-THPC), N-aspartyl chlorin e6 (NPe6), and tin ethyl etiopurpurin (SnET2)), pheophorbides (e.g., 2-(1-hexyloxyethyl)-2-devinyl pyropheophorbide (HPPH)), bacteriopheophorbides (e.g., bacteriochlorphylla, WSTO9 and WST11), Texaphyrins (e.g., motexafin lutetium (Lu-Tex)), and phthalocyanines (PCs) (e.g., aluminum phthalocyanine tetrasulfonate (AlPcS4) and silicon phthalocyanine (Pc4)). In some embodiments, the PDT sensitizer can include cationic zinc ethynylphenyl porphyrin. Although porphyrinoid structures comprise a majority of photosensitizers, several non-porphyrin chromogens exhibit photodynamic activity. These compounds include anthraquinones, phenothiazines, xanthenes, cyanines, and curcuminoids. Alternatively, the photosensitizer can include indocyanine green (ICG).

In some embodiments, a theranostic agent or therapeutic agent described herein can include a phthalocyanine compound. Phthalocyanines, hereinafter also abbreviated as “Pcs”, are a group of photosensitizer compounds having the phthalocyanine ring system. Phthalocyanines are azaporphyrins consisting of four benzoindole groups connected by nitrogen bridges in a 16-membered ring of alternating carbon and nitrogen atoms (i.e., C32H16N8) which form stable chelates with metal and metalloid cations. In these compounds, the ring center is occupied by a metal ion (either a diamagnetic or a paramagnetic ion) that may, depending on the ion, carry one or two ligands. In addition, the ring periphery may be either unsubstituted or substituted. Phthalocyanines strongly absorb clinically useful red or near IR radiation with absorption peaks falling between about 600 and 810 nm, which potentially allows deep tissue penetration by the light. The synthesis and use of a wide variety of phthalocyanines in photodynamic therapy is described in International Publication WO 2005/099689.

In some embodiments, the phthalocyanine compound is Pc4. Pc4 is relatively photostable and virtually non-toxic. In some embodiments, the phthalocyanine compound is an analog of the PDT photosensitizing drug Pc4 found to be effective in targeted bioimaging and targeted PDT of cancer in a subject, see for example, U.S. Pat. No. 9,889,199, the contents of which are hereby incorporated by reference. In some embodiments, the Pc4 analog can include Pc413.

In other embodiments, the therapeutic agent or theranostic agent is a nanobubble that is directly or indirectly linked via the peptide or peptidomimetic spacer to the targeting peptide. The nanobubble can have a membrane that defines at least one internal void, which includes at least one gas and, optionally, at least one therapeutic agent that is contained within the membrane or conjugated to the membrane of each nanobubble. The therapeutic agent can include, for example, at least one chemotherapeutic agent, anti-proliferative agent, biocidal agent, biostatic agent, or anti-microbial agent.

An agent comprising the targeting peptide, peptide or peptidomimetic spacer, and nanobubble can be administered to a subject with cancer. The targeting peptide can bind to a target cancer cell, and the nanobubbles can have a size, diameter, and/or composition that facilitates internalization of the cancer cell targeted nanobubbles by the target cancer cell upon binding of the targeting peptide to the cancer cell. Following administration of the targeted nanobubbles to the subject, cell targeted nanobubbles internalized into the target cell can be insonated with ultrasound energy effective to promote inertial cavitation of the internalized nanobubbles and apoptosis and/or necrosis of the target cancer cell and/or release of a therapeutic agent, such as a chemotherapeutic, from the nanobubbles to the cancer cells.

In other embodiments the therapeutic agent linked to a peptide or peptidomimetic spacer and a targeting peptide can include an anti-cancer or an anti-proliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be directly or indirectly linked to a targeting peptide in an agent described herein include Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon α-2a; interferon α-2b; interferon α n1; interferon α-n3; interferon β-I a; interferon γ-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer therapeutic agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Other anti-cancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Still other anti-cancer therapeutic agents include alkylating agents, such as nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.), antimetabolites, such as folic acid analog (e.g., methotrexate), or pyrimidine analogs (e.g., fluorouracil, floxouridine, Cytarabine), purine analogs (e.g., mercaptopurine, thioguanine, pentostatin, vinca alkaloids (e.g., vinblastin, vincristine), epipodophyllotoxins (e.g., etoposide, teniposide), platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In some embodiments, cytotoxic compounds are included in a agent described herein. Cytotoxic compounds include small-molecule drugs such as doxorubicin, mitoxantrone, methotrexate, and pyrimidine and purine analogs, referred to herein as antitumor agents.

The agents including a targeting peptide, spacer, and therapeutic agent described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The agents including a targeting peptide, peptide or peptidomimetic spacer, and therapeutic agent described herein can be administered alone as a monotherapy, or in conjunction with or in combination with one or more additional therapeutic agents. For example, the agent including a targeting peptide linked to a therapeutic agent described herein can be administered to the subject prior to, during, or post administration of an additional therapeutic agent and the distribution of metastatic cells can be targeted with the therapeutic agent. The agent can be administered to the animal as part of a pharmaceutical composition comprising the agent and a pharmaceutically acceptable carrier or excipient and, optionally, one or more additional therapeutic agents. The agent including a targeting peptide, peptide or peptidomimetic spacer, and therapeutic agent described herein and additional therapeutic agent can be components of separate pharmaceutical compositions, which can be mixed together prior to administration or administered separately. The agent including targeting peptide, peptide or peptidomimetic spacer, and therapeutic agent described herein, for example, be administered in a composition containing the additional therapeutic agent, and thereby, administered contemporaneously with the agent. Alternatively, the agent including a targeting peptide, peptide or peptidomimetic spacer, and therapeutic agent described herein can be administered contemporaneously, without mixing (e.g., by delivery of the agent on the intravenous line by which the therapeutic agent is also administered, or vice versa). In another embodiment, the agent including a targeting peptide, peptide or peptidomimetic spacer, and therapeutic agent described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The agent including a targeting peptide, peptide or peptidomimetic spacer, and therapeutic agent described herein (or composition containing the agent) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the agent and/or an additional therapeutic agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the detectable moiety, therapeutic or theranostic agent in the subject, the agent can be administered between, for example, once a day or once a week.

For example, the administration of the agent and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The agent including a targeting peptide, peptide or peptidomimetic spacer, and therapeutic agent described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of disclosed agent including a targeting peptide, peptide or peptidomimetic spacer, and therapeutic agent described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the agent including the targeting peptide, peptide or peptidomimetic spacer, and therapeutic agent described herein can also depend on the disease state or condition being treated along with the clinical factors and the route of administration of the compound.

The disclosed agent and/or additional therapeutic agent described herein can be administered to the subject in conjunction with an acceptable pharmaceutical carrier or diluent as part of a pharmaceutical composition for therapy. Formulation of the compound to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

The following example is included to demonstrate preferred embodiments.

### Example

### Methods

### Peptide Synthesis and Conjugation

SBK targeting peptides (e.g., GEGDDFNWEQVNTLTKPTSD (SEQ ID NO: 5)) and Scrambled (GTQDETGNFDWPVSEDLNKT (SEQ ID NO: 47)) peptides were either were synthesized on a synthesizer at Case Western Reserve University or were purchased from PolyPeptide Group (San Diego, Calif.). An N-terminal glycine or glycine/serine spacer was added to peptides during synthesis. After synthesis, the N-terminal glycine residue of each peptide spacer was specifically coupled to Texas Red (TR)-X (single isomer) which has a five-carbon spacer between the succinimide group that couples to the N-terminal amine and the fluorophore. Alternatively, peptides were coupled to Indocyanine green (ICG). Various lengths of amino acids spacer can also be added during peptide synthesis. N-terminal cysteine residues can also be used for conjugation.

### Cell Culture and Heterotopic Xenograft Flank Tumor Implants

The human U87-MG and LN-229 glioma cell line was purchased from American Type Culture Collection (Manassas, Va., USA) and cultured. NIH athymic nude mice (5-8 weeks and 20-25 g on arrival; NCI-NIH) were maintained at the Athymic Animal Core Facility at Case Western Reserve University according to institutional policies. All animal protocols were approved by the Institutional Animal Care and Use Committee (IACUC). The cells were diluted in a 1:1 mixture of PBS and BD MATRIGEL Matrix (BD Biosciences, Franklin Lakes, N.J., USA), and injected into the right flank of nude athymic mice (NCr-nu/+, NCr-nu/nu, 20-25 g each). The Matrigel-cell mixture was loaded into a 1-ml syringe fitted with a 26-gauge needle and kept on ice. The mixture was injected subcutaneously in the right flank region of the mouse. Flank tumors were grown for 2 to 3 weeks. Each flank was implanted with 1.4-2×10⁶ cells. To correlate tumor position with GFP fluorescence, mice were imaged using the Perkin-Elmer MAESTRO FLEX In vivo Imaging System. For in vivo analyses, the mice were anesthetized with inhaled isoflurane/oxygen and imaged. For ex vivo analyses, mice were sacrificed by decapitation. Flank tumors were then excised and imaged.

### In Vivo Imaging of Flank Tumors

Nude mice with heterotopic (flank) tumors were typically imaged 3 to 6 weeks following tumor initiation. Nude mice with orthotopic (intracranial) tumors were typically imaged 7 to 14 days after tumor cell implantation. Fluorophore-conjugated PTPρpeptides were diluted to 100 μM-200 μM and injected through a lateral tail vein to administer the desired dose of the agent, generally between 100-400 nmol/kg. In animals where green fluorescent protein (GFP)-expressing tumor cells were used to facilitate monitoring tumor growth and/or migration, fluorophores with limited spectral overlap to GFP were utilized. In vivo and ex vivo images of specific tissues were obtained using the IVIS Spectrum In Vivo Imaging System (Perkin Elmer, Waltham, Mass., USA) with the manufacturers recommended excitation and emission filters for a given fluorophore, along with the built-in auto-exposure feature. The following are some filter pair examples used for different fluorophores: 465/520 for GFP; Texas Red, 570/620; Cy5, 640/680; IR800CW, 745/800; Indocyanine Green (ICG), 745/820. Background images were acquired prior to injecting any fluorescent peptides to allow baseline measurements. Animals were imaged ten minutes after injecting the peptides, and at ten minutes intervals, as needed, up to 2 h. For some fluorescent peptides, additional in vivo images were acquired between 8 and 24 h. Following the final in vivo image, mice were euthanized. Flank tumors or intact brains with intracranial tumors, along with other organs of interest were excised and imaged ex vivo. Data were imported into LivingImage software (PerkinElmer) for image analyses and binning was set to 1. Region of Interest (ROI) analysis was used to examine the fluorescent signal obtained in either specific locations, using ROIs of defined size, or in the entire mouse or organ, using an ROI outlining the body or organ. Average radiant intensity units, calculated by the LivingImage software, were obtained for each ROI. Each fluorescent PTPρpeptide was tested on a minimum of three tumor-bearing animals Statistical analyses were performed using Microsoft Excel and an unpaired Student's t test. Spectral fluorescence images were also obtained using the Maestro FLEX In vivo Imaging System using the appropriate filters for GFP (tumor; excitation=445-490 nm, emission=515 nm long-pass filter, acquisition settings=500-720 in 10-nm steps), TR (peptide; excitation=575-605 nm, emission=645 nm; acquisition settings=630-850 in 10-nm steps), or Alexa-750 (peptide; excitation=671-705 nm, emission=750 nm long-pass filter, acquisition settings=730-950 in 10-nm steps). Acquisition settings were 53 milliseconds for GFP and 1000 milliseconds for either TR or Alexa-750-labeled peptide. Before peptide injection, background images were acquired through the skin to provide autofluorescence spectra. After peptide injection, fluorescence images were acquired at 5- to 15-minute intervals during 2 to 3 hours. The multispectral fluorescence images were background subtracted and unmixed, using Maestro software (Cambridge Research & Instrumentation, Inc, Woburn, Mass.), to spectrally separate the autofluorescence animal signal from the peptide signals. Regions of interest (ROIs) were selected over the tumor or nontumor skin. Pixel values for the peptide signal, in photons measured at the surface of the animal, were determined within these ROIs. Higher pixel values corresponded to presence of tumor. Pixel values in the tumor ROIs were normalized to the nontumor ROI and peptide concentration and were then plotted. Each PTPρpeptide was tested on a minimum of three animals containing flank tumors. Statistical analyses were performed using Microsoft Excel and an unpaired Student's t test.

### Ex Vivo Imaging of Tumors

Ex vivo imaging was performed after injection of the SBK peptide agents in live mice bearing tumors to allow for clearance of unbound agent. After various intervals for clearance of unbound agent, the animals were sacrificed and brains were excised for ex vivo optical imaging and histology. Imaging with either the Spectrum or the MAESTRO FLEX In vivo Imaging System (Cambridge Research & Instrumentation (CRi), Woburn, Mass.) was performed as previously described. Excised whole brains were imaged using filters appropriate for GFP (tumor cells) as described and various fluorophores.

### Orthotopic Xenograft Intracranial Tumors

NIH athymic nude female mice (NCr-nu/+, NCr-nu/nu) were bred in the Athymic Animal Core Facility and housed in the Case Center for Imaging Research at Case Western Reserve University according to the Institutional Animal Care and Use Committee approved animal protocols. Human U-87 MG glioma cells were obtained from American Type Culture Collection. CNS-1 rodent glioma cells were obtained from Mariano S. Viapiano. SJ-GBM2 cells were derived from a post-mortem 5-year old female patient with GBM and were obtained from Children's Oncology Group Cell Line & Xenograft Repository. Where indicated, cells were infected with lentivirus to express green fluorescent protein (GFP) or m-Cherry and intracranial implantation of tumor cells was performed as described. Briefly, mice 6 to 7 weeks of age were anesthetized and fitted into a stereotaxic rodent frame (David Kopf Instruments, Tujunga, Calif.). A small burr hole was made 0.7 mm anterior and 2 mm lateral from bregma. Cells were harvested for intracranial implantation and deposited into the right striatum at a depth of −3 mm from the dura using a 10 μL syringe. A total of 2×10⁵ U-87 MG cells, 4.5×10⁴ CNS-1 cells, or 3×10⁵ SJ-GBM2 cells were injected. The needle was slowly withdrawn, and the incision was closed with sutures. Mice were imaged as described below then sacrificed 8-21 days post tumor implant. Brain tissue was collected for imaging and histological processing.

### In Vivo Labeling of Intracranial Tumors

Nude mice with intracranial tumors were imaged at 9 to 12 days after GBM cell implant. Fluorophore conjugated PTPρpeptides were injected through the tail vein. After a 25-minute incubation for clearance of unbound PTPρpeptide, the animals were sacrificed, and the brains were removed and imaged either whole or sliced into coronal sections at 1-mm intervals. Individual brain sections containing tumor were placed on a black slide and examined using either the Spectrum or Maestro FLEX In vivo Imaging System as described above. Untreated brains containing intracranial tumors were used to provide autofluorescence spectra. ROIs were selected over the tumor region in each brain slice. Pixel values for the peptide signal, in photons measured from the slice, were determined within these ROIs. The multispectral fluorescence images were background-subtracted and analyzed using the Maestro software as described previously. Statistical analyses were performed using Microsoft Excel and an unpaired Student's t test.

### Results

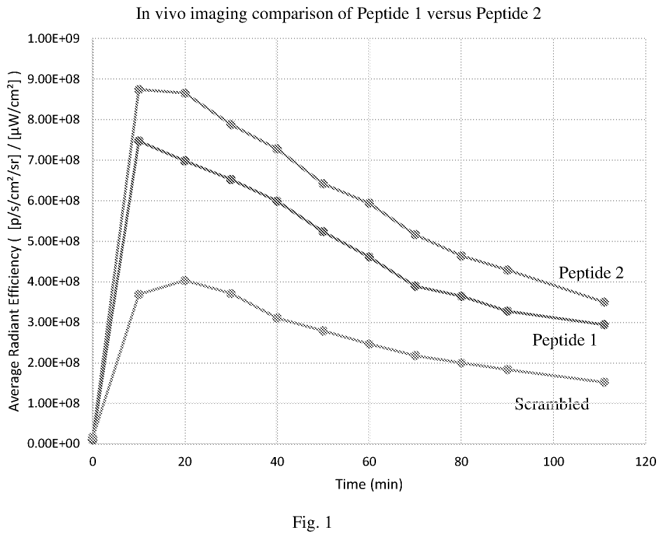

FIGS. 1 to 7 compare binding and in vivo average radiant efficiency of various imaging agents administered to mice with heterotopic xenograft flank U87 tumor implants. The imaging agents include an SBK targeting peptide that is linked to a fluorophore with a polyglycine or glycine/serine spacer or a control scramble peptide that is linked directly to a fluorophore or with on polyglycine or glycine/serine spacers.

FIG. 1 illustrates plots showing in vivo average radiant efficiency of a first agent (Peptide 1, which includes SBK linked to a fluorophore without a peptide spacer), a second agent (Peptide 2, which includes SBK linked to a fluorophore with a polyglycine peptide spacer), and a control agent (Scrambled, which includes a scrambled peptide that is linked to a fluorophore with the polyglycine peptide spacer) administered to mice with heterotopic xenograft flank U87 tumor implant. In vivo imaging of the flank tumors of the mice shows the second agent, which includes the polyglycine peptide spacer, has an increased average radiant efficiency compared to the first agent, without a spacer, and the control agent, which includes the polyglycine peptide spacer.

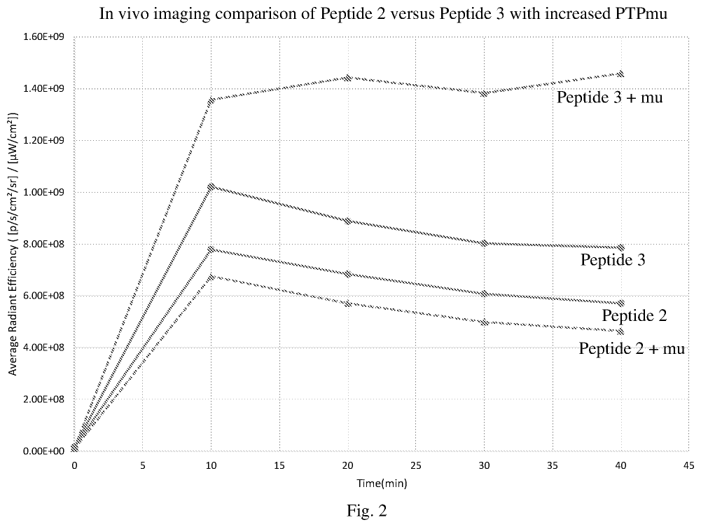

FIG. 2 illustrates plots showing the in vivo average radiant efficiency of the second agent (Peptide 2, which includes SBK linked to a fluorophore with a polyglycine spacer) and a third agent (Peptide 3, which includes SBK linked to a flurophore with a glycine/serine spacer) administered to mice with heterotopic xenograft U87 flank tumor implants or mice with heterotopic xenograft flank U87 tumor implants overexpressing PTPmu. In vivo imaging of the U87 flank tumors and the U87 flank tumors overexpressing PTPmu shows the third agent, which includes the glycine/serine peptide spacer, has an increased average radiant efficiency compared to the second agent, with the polyglycine spacer, in both the U87 flank tumors and the U87 flank tumors overexpressing PTPmu.

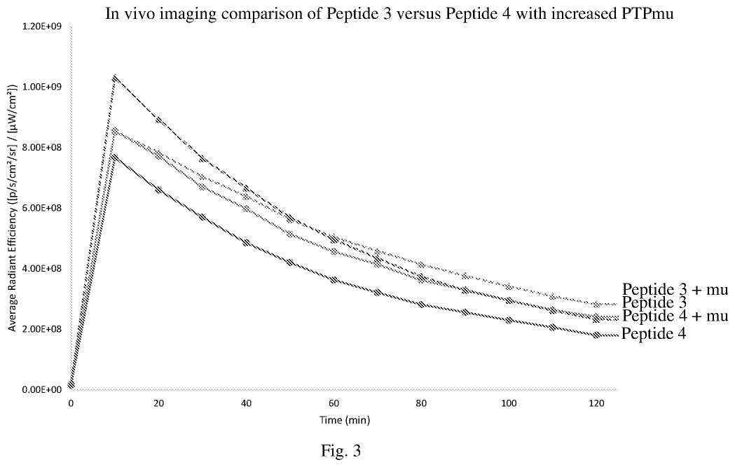

FIG. 3 illustrates plots showing the in vivo average radiant efficiency of the third agent (Peptide 3, which includes SBK linked to a fluorophore with a glycine/serine peptide spacer) and a fourth agent (Peptide 4, which includes SBK linked to a fluorophore with a second glycine/serine spacer) administered to mice with heterotopic xenograft U87 flank tumor implants or mice with heterotopic xenograft flank U87 tumor implants overexpressing PTPmu.

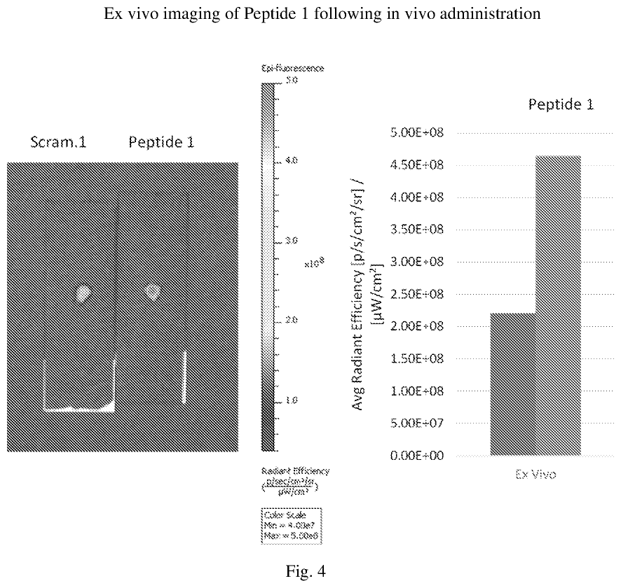

FIG. 4 illustrates ex vivo images and a graph showing average radiant efficiency of the first agent (Peptide 1, which includes SBK linked to a fluorophore without a peptide spacer) and a control agent (Scrambled 1, which includes a scrambled peptide that is linked to a fluorophore with the polyglycine peptide spacer of Peptide 1) following in vivo administration to mice with heterotopic xenograft U87 flank tumor implants.

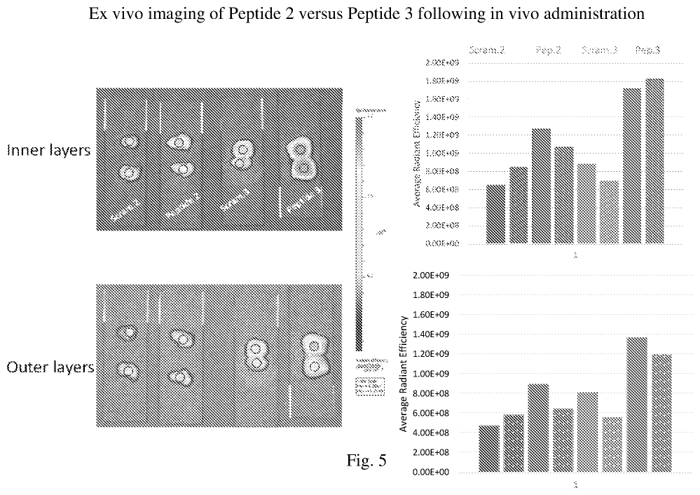

FIG. 5 illustrates ex vivo images and graphs showing average radiant efficiency of the second agent (Peptide 2, which includes SBK linked to a fluorophore with a polyglycine peptide spacer), the third agent (Peptide 3, which includes SBK linked to a fluorophore with a glycine/serine peptide spacer), and control agents (Scrambled 2, which includes a scrambled peptide that is linked to a fluorophore with the polyglycine spacer of Peptide 2, and Scrambled 3, which includes a scrambled peptide that is linked to a fluorophore with the glycine/serine spacer of Peptide 3) following in vivo administration to mice with heterotopic xenograft U87 flank tumor implants.

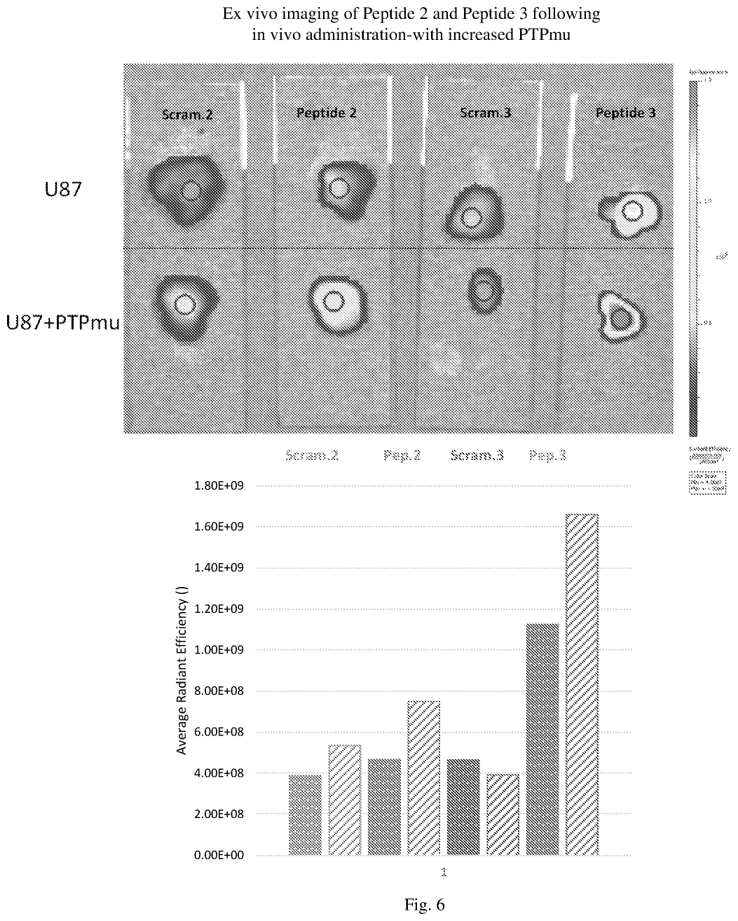

FIG. 6 illustrates ex vivo images and a graph showing average radiant efficiency of the second agent (Peptide 2, which includes SBK linked to a fluorophore with a polyglycine peptide spacer), the third agent (Peptide 3, which includes SBK linked to a fluorophore with a glycine/serine peptide spacer), and control agents (Scrambled 2, which includes a scrambled peptide that is linked to a fluorophore with the polyglycine spacer of Peptide 2, and Scrambled 3, which includes a scrambled peptide that is linked to a fluorophore with the glycine/serine spacer of Peptide 3) following in vivo administration to mice with heterotopic xenograft U87 flank tumor implants overexpressing PTPmu.

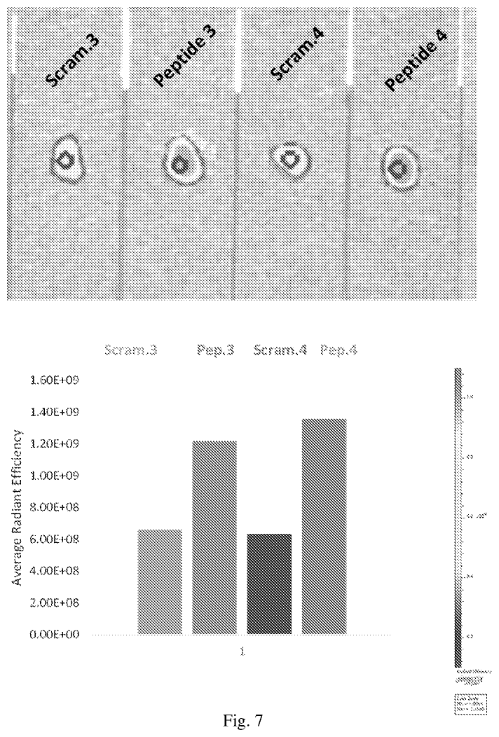

FIG. 7 illustrates ex vivo images and a graph showing average radiant efficiency of the third agent (Peptide 3, which includes SBK linked to a fluorophore with a glycine/serine peptide spacer), the fourth agent (Peptide 4, which includes SBK linked to a fluorophore with a second glycine/serine spacer), and control agents (Scrambled 3, which includes a scrambled peptide that is linked to a fluorophore with the glycine/serine spacer of Peptide 3, and Scrambled 4, which includes a scrambled peptide that is linked to a fluorophore with the glycine/serine spacer of Peptide 4) following in vivo administration to mice with heterotopic xenograft U87 flank tumor implants overexpressing PTPmu.

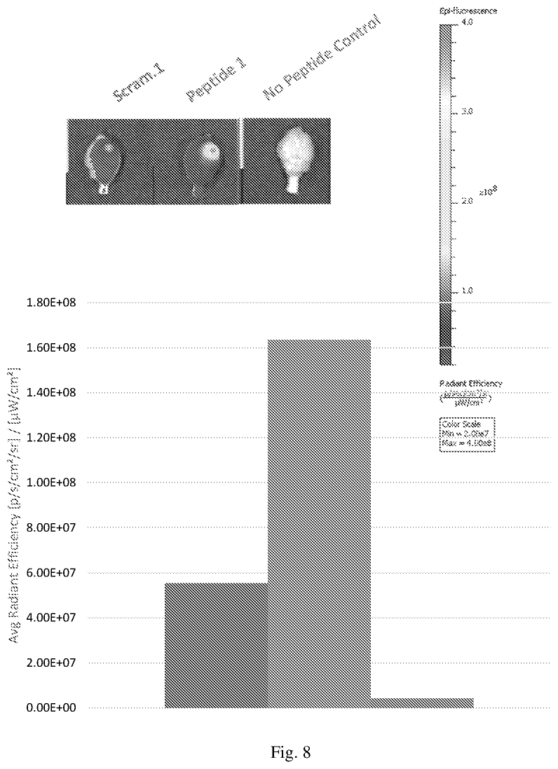

FIGS. 8 to 12 compare binding and in vivo average radiant efficiency of various imaging agents administered to mice with orthotopic xenograft U87 intracranial tumors. The imaging agents include an SBK targeting peptide that is linked to a fluorophore with a polyglycine or glycine/serine spacer or a control scrambled peptide that is linked directly to a fluorophore or with on polyglycine or glycine/serine spacers.

FIG. 8 illustrates ex vivo images and a graph showing average radiant efficiency of the first agent (Peptide 1, which includes SBK linked to a fluorophore without a peptide spacer) compared to a control agent (Scrambled, which includes a scrambled peptide that is linked to a fluorophore with the polyglycine peptide spacer) following in vivo administration to mice with orthotopic xenograft U87 intracranial tumors

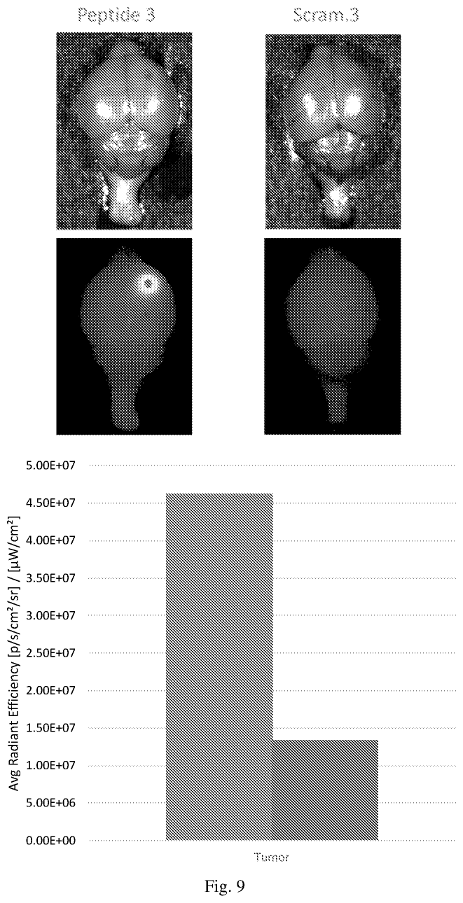

FIG. 9 illustrates ex vivo images and a graph showing average radiant efficiency of the third agent (Peptide 3, which includes SBK linked to a fluorophore with a glycine/serine peptide spacer) compared to a control agent (Scrambled 3, which includes a scrambled peptide that is linked to a fluorophore with the glycine/serine spacer of Peptide 3) following in vivo administration to mice with orthotopic xenograft U87 intracranial tumors.

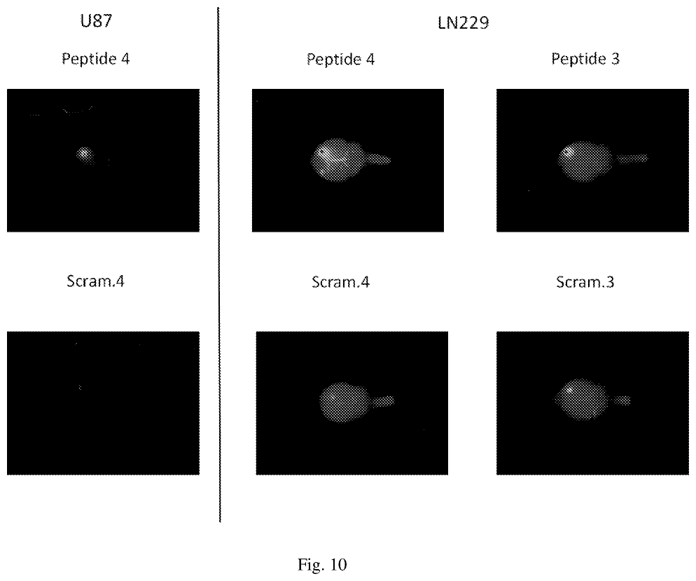

FIG. 10 illustrates ex vivo images of orthotopic xenograft U87 intracranial tumors or orthotopic xenograft LN229 intracranial tumors following in vivo administration to mice of the third agent (Peptide 3, which includes SBK linked to a fluorophore with a glycine/serine peptide spacer), the fourth agent (Peptide 4, which includes SBK linked to a fluorophore with a second glycine/serine spacer), and control agents (Scrambled 3, which includes a scrambled peptide that is linked to a fluorophore with the glycine/serine spacer of Peptide 3, and Scrambled 4, which includes a scrambled peptide that is linked to a fluorophore with the glycine/serine spacer of Peptide 4).

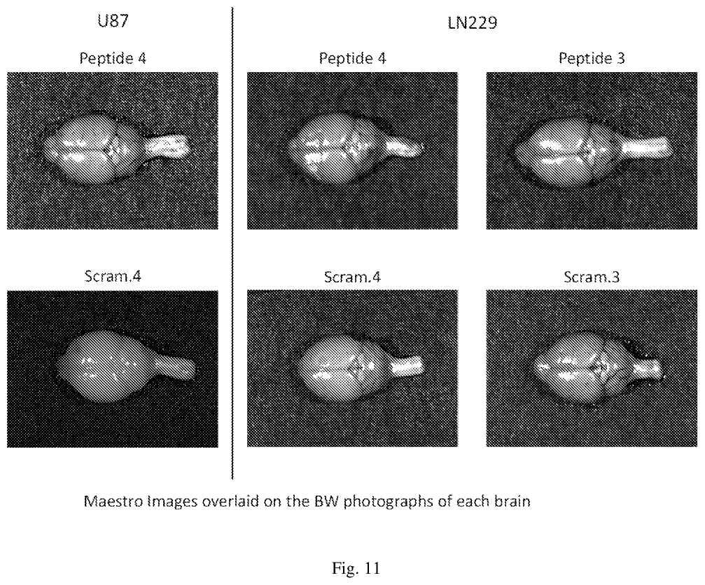

FIG. 11 illustrates ex vivo maestro images overlaid on black and white photographs of the brain following in vivo administration to mice of the third agent (Peptide 3, which includes SBK linked to a fluorophore with a glycine/serine peptide spacer), the fourth agent (Peptide 4, which includes SBK linked to a fluorophore with a second glycine/serine spacer), and control agents (Scrambled 3, which includes a scrambled peptide that is linked to a fluorophore with the glycine/serine spacer of Peptide 3, and Scrambled 4, which includes a scrambled peptide that is linked to a fluorophore with the glycine/serine spacer of Peptide 4).

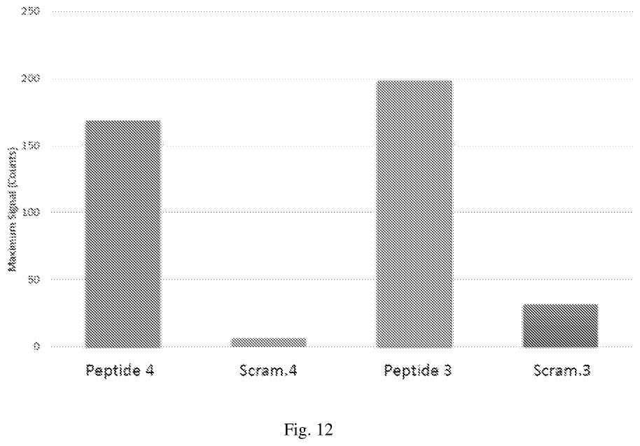

FIG. 12 illustrates a graph showing maximum signal intensity of the third agent (Peptide 3, which includes SBK linked to a fluorophore with a glycine/serine peptide spacer), the fourth agent (Peptide 4, which includes SBK linked to a fluorophore with a second glycine/serine spacer), and control agents (Scrambled 3, which includes a scrambled peptide that is linked to a fluorophore with the glycine/serine spacer of Peptide 3, and Scrambled 4, which includes a scrambled peptide that is linked to a fluorophore with the glycine/serine spacer of Peptide 4) following in vivo administration to mice with orthotopic xenograft U87 intracranial tumors.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1. An agent comprising:
a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment;
at least one of a detectable moiety, therapeutic agent, or theranostic agent; and
a peptide or peptidomimetic spacer that directly or indirectly links the targeting peptide to the at least one of the detectable moiety, therapeutic agent, or theranostic agent, wherein the spacer has a length and structure effective to at least maintain or preserve binding affinity of the linked targeting peptide to the proteolytically cleaved extracellular fragment and activity of the at least one of the linked detectable moiety, therapeutic agent, or a theranostic agent.

2. The agent of claim 1, for use in detecting, monitoring, and/or imaging cancer cells and/or cancer cell metastasis, migration, dispersal, and/or invasion, and/or for treating cancer in a subject.

3. The agent claim 1, being configured for in vivo administration to a subject or ex vivo administration to biological sample of the subject.

4. The agent of claim 1, wherein the spacer includes natural and/or non-natural amino acids.

5. The agent of claim 1, wherein the spacer includes at least 3 natural or non-natural amino acids.

6. The agent of claim 1, wherein the spacer has a length of 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids.

7. The agent of claim 1, wherein the spacer includes at least about 50%, at least about 60%, at least about 70%, at least about 80%, or at least about 90% glycine and/or serine residues

8. The agent of claim 1, wherein the spacer includes at least 50%, at least 60%, at least 70%, or at least 80% glycine residues.

9. The agent of claim 1, wherein the spacer is a polyglycine or glycine/serine spacer.

10. The agent of claim 1, wherein spacer comprises the amino acid sequence of at least one of (GS)a, (GGS)b, or (GGGS)c, or (GGGGS)d and wherein a, b, c, and d are each independently 2, 3, 4, 5, or 6.

11. The agent of claim 1, wherein the spacer has an amino acid sequence of GGG (SEQ ID NO: 9), GGGG (SEQ ID NO: 10), GGGGG (SEQ ID NO: 11), GGGGGG (SEQ ID NO: 12), GGGGGGG (SEQ ID NO: 13), GGGGGGGG (SEQ ID NO: 14), GGGGGGGGG (SEQ ID NO: 15), GSGS (SEQ ID NO: 16), GSGSGS (SEQ ID NO: 17), GSGSGSGS (SEQ ID NO: 18), GSGSGSGSGS (SEQ ID NO: 19), GGSGGS (SEQ ID NO: 20), GGSGGSGGS (SEQ ID NO: 21), GGSGGSGGSGGS (SEQ ID NO: 22), GGGSGGGS (SEQ ID NO: 23), GGGSGGGSGGGS (SEQ ID NO: 24), GGGSGGGSGGGSGGGS (SEQ ID NO: 25), GGGGSGGGGS (SEQ ID NO: 26), or GGGGSGGGGSGGGGS (SEQ ID NO: 27).

12. The agent of claim 1, further including at least one coupling agent that links the spacer to the targeting peptide and/or the at the at least one of the detectable moiety, therapeutic agent, or theranostic agent.

13. The agent of claim 1, wherein the cell adhesion molecule comprises a cell surface receptor protein tyrosine phosphatase (PTP) type IIb.

14. The agent of claim 1, wherein the extracellular fragment comprises the amino acid sequence of SEQ ID NO: 2 and the targeting peptide comprising a polypeptide that specifically binds to and/or complexes to SEQ ID NO: 2.

15. The agent of claim 1, wherein the targeting peptide comprises a polypeptide having an amino acid sequence that has at least 80% sequence identity to about 10 to about 50 consecutive amino acids of SEQ ID NO: 3.

16. The agent of claim 1, wherein the targeting peptide comprises a polypeptide having an amino acid sequence selected from the group consisting of SEQ ID NO: 4, SEQ ID NO: 5, SEQ ID NO: 6, SEQ ID NO: 7, and SEQ ID NO: 8.

17. The agent of claim 1, wherein the detectable moiety comprises a chelating agent, contrast agent, imaging agent, radiolabel, semiconductor particle, nanoparticle, nanobubble, or nanochain.

18. The agent of claim 1, wherein the detectable moiety is detectable by at least one of magnetic resonance imaging (MRI), positron emission tomography (PET) imaging, computer tomography (CT) imaging, gamma imaging, near infrared imaging, or fluorescent imaging.

19. The agent of claim 1, wherein the theranostic or therapeutic agent comprises at least one of a photosensitizer, ultrasound sensitizer, thermal sensitizer, radiosensitizer, radiotherapeutic, chemotherapeutic, or immunotherapeutic.

20-27. (canceled)


SEQUENCE LISTING (47 sequences)

SEQ ID NO: 1 - protein, 1465 aa, Homo sapiens [sequence omitted]

SEQ ID NO: 2 - protein, 477 aa, Homo sapiens
MRGLGTCLATLAGLLLTAAGETFSGGCLFDEPYSTCGYSQSEGDDFNWEQVNTLTKPTSDPWMPSGSFMLVNASGRPEGQRAHLLLPQLKENDTHCIDFHYFVSSKSNSPPGLLNVYVKVNNGPLGNPIWNISGDPTRTWNRAELAISTFWPNFYQVIFEVITSGHQGYLAIDEVKVLGHPCTRTPHFLRIQNVEVNAGQFATFQCSAIGRTVAGDRLWLQGIDVRDAPLKEIKVTSSRRFIASFNVVNTTKRDAGKYRCMIRTEGGVGISNYAELVVKEPPVPIAPPQLASVGATYLWIQLNANSINGDGPIVAREVEYCTASGSWNDRQPVDSTSYKIGHLDPDTEYEISVLLTRPGEGGTGSPGPALRTRTKCADPMRGPRKLEVVEVKSRQITIRWEPFGYNVTRCHSYNLTVHYCYQVGGQEQVREEVSWDTENSHPQHTITNLSPYTNVSVKLILMNPEGRKESQELIVQT

SEQ ID NO: 3 - protein, 280 aa, Homo sapiens
MRGLGTCLATLAGLLLTAAGETFSGGCLFDEPYSTCGYSQSEGDDFNWEQVNTLTKPTSDPWMPSGSFMLVNASGRPEGQRAHLLLPQLKENDTHCIDFHYFVSSKSNSPPGLLNVYVKVNNGPLGNPIWNISGDPTRTWNRAELAISTFWPNFYQVIFEVITSGHQGYLAIDEVKVLGHPCTRTPHFLRIQNVEVNAGQFATFQCSAIGRTVAGDRLWLQGIDVRDAPLKEIKVTSSRRFIASFNVVNTTKRDAGKYRCMIRTEGGVGISNYAELVVKE

SEQ ID NO: 4 - protein, 20 aa, Artificial Sequence
  Synthetic peptide
ETFSGGCLFDEPYSTCGYSQ

SEQ ID NO: 5 - protein, 20 aa, Artificial Sequence
  Synthetic peptide
GEGDDFNWEQVNTLTKPTSD

SEQ ID NO: 6 - protein, 19 aa, Artificial Sequence
  Synthetic peptide
TPHFLRIQNVEVNAGQFAT

SEQ ID NO: 7 - protein, 18 aa, Artificial Sequence
  Synthetic peptide
GIDVRDAPLKEIKVTSSR

SEQ ID NO: 8 - protein, 21 aa, Artificial Sequence
  Synthetic Construct
CGEGDDFNWEQVNTLTKPTSD

SEQ ID NO: 9 - protein, 3 aa, Artificial Sequence
  Synthetic Construct
GGG

SEQ ID NO: 10 - protein, 4 aa, Artificial Sequence
  Synthetic Construct
GGGG

SEQ ID NO: 11 - protein, 5 aa, Artificial Sequence
  Synthetic Construct
GGGGG

SEQ ID NO: 12 - protein, 6 aa, Artificial Sequence
  Synthetic Construct
GGGGGG

SEQ ID NO: 13 - protein, 7 aa, Artificial Sequence
  Synthetic Construct
GGGGGGG

SEQ ID NO: 14 - protein, 8 aa, Artificial Sequence
  Synthetic Construct
GGGGGGGG

SEQ ID NO: 15 - protein, 9 aa, Artificial Sequence
  Synthetic Construct
GGGGGGGGG

SEQ ID NO: 16 - protein, 4 aa, Artificial Sequence
  Synthetic Construct
GSGS

SEQ ID NO: 17 - protein, 6 aa, Artificial Sequence
  Synthetic Construct
GSGSGS

SEQ ID NO: 18 - protein, 8 aa, Artificial Sequence
  Synthetic Construct
GSGSGSGS

SEQ ID NO: 19 - protein, 10 aa, Artificial Sequence
  Synthetic Construct
GSGSGSGSGS

SEQ ID NO: 20 - protein, 6 aa, Artificial Sequence
  Synthetic Construct
GGSGGS

SEQ ID NO: 21 - protein, 9 aa, Artificial Sequence
  Synthetic Construct
GGSGGSGGS

SEQ ID NO: 22 - protein, 12 aa, Artificial Sequence
  Synthetic Construct
GGSGGSGGSGGS

SEQ ID NO: 23 - protein, 8 aa, Artificial Sequence
  Synthetic Construct
GGGSGGGS

SEQ ID NO: 24 - protein, 12 aa, Artificial Sequence
  Synthetic Construct
GGGSGGGSGGGS

SEQ ID NO: 25 - protein, 16 aa, Artificial Sequence
  Synthetic Construct
GGGSGGGSGGGSGGGS

SEQ ID NO: 26 - protein, 10 aa, Artificial Sequence
  Synthetic Construct
GGGGSGGGGS

SEQ ID NO: 27 - protein, 15 aa, Artificial Sequence
  Synthetic Construct
GGGGSGGGGSGGGGS

SEQ ID NO: 28 - protein, 23 aa, Artificial Sequence
  Synthetic Construct
GGGGEGDDFNWEQVNTLTKPTSD

SEQ ID NO: 29 - protein, 24 aa, Artificial Sequence
  Synthetic Construct
GGGGGEGDDFNWEQVNTLTKPTSD

SEQ ID NO: 30 - protein, 25 aa, Artificial Sequence
  Synthetic Construct
GGGGGGEGDDFNWEQVNTLTKPTSD

SEQ ID NO: 31 - protein, 26 aa, Artificial Sequence
  Synthetic Construct
GGGGGGGEGDDFNWEQVNTLTKPTSD

SEQ ID NO: 32 - protein, 27 aa, Artificial Sequence
  Synthetic Construct
GGGGGGGGEGDDFNWEQVNTLTKPTSD

SEQ ID NO: 33 - protein, 28 aa, Artificial Sequence
  Synthetic Construct
GGGGGGGGGEGDDFNWEQVNTLTKPTSD

SEQ ID NO: 34 - protein, 29 aa, Artificial Sequence
  Synthetic Construct
GGGGGGGGGGEGDDFNWEQVNTLTKPTSD

SEQ ID NO: 35 - protein, 24 aa, Artificial Sequence
  Synthetic Construct
GSGSGEGDDFNWEQVNTLTKPTSD

SEQ ID NO: 36 - protein, 26 aa, Artificial Sequence
  Synthetic Construct
GSGSGSGEGDDFNWEQVNTLTKPTSD

SEQ ID NO: 37 - protein, 28 aa, Artificial Sequence
  Synthetic Construct
GSGSGSGSGEGDDFNWEQVNTLTKPTSD

SEQ ID NO: 38 - protein, 30 aa, Artificial Sequence
  Synthetic Construct
GSGSGSGSGSGEGDDFNWEQVNTLTKPTSD

SEQ ID NO: 39 - protein, 26 aa, Artificial Sequence
  Synthetic Construct
GGSGGSGEGDDFNWEQVNTLTKPTSD

SEQ ID NO: 40 - protein, 29 aa, Artificial Sequence
  Synthetic Construct
GGSGGSGGSGEGDDFNWEQVNTLTKPTSD

SEQ ID NO: 41 - protein, 32 aa, Artificial Sequence
  Synthetic Construct
GGSGGSGGSGGSGEGDDFNWEQVNTLTKPTSD

SEQ ID NO: 42 - protein, 28 aa, Artificial Sequence
  Synthetic Construct
GGGSGGGSGEGDDFNWEQVNTLTKPTSD

SEQ ID NO: 43 - protein, 32 aa, Artificial Sequence
  Synthetic Construct
GGGSGGGSGGGSGEGDDFNWEQVNTLTKPTSD

SEQ ID NO: 44 - protein, 36 aa, Artificial Sequence
  Synthetic Construct
GGGSGGGSGGGSGGGSGEGDDFNWEQVNTLTKPTSD

SEQ ID NO: 45 - protein, 30 aa, Artificial Sequence
  Synthetic Construct
GGGGSGGGGSGEGDDFNWEQVNTLTKPTSD

SEQ ID NO: 46 - protein, 35 aa, Artificial Sequence
  Synthetic Construct
GGGGSGGGGSGGGGSGEGDDFNWEQVNTLTKPTSD

SEQ ID NO: 47 - protein, 20 aa, Artificial Sequence
  Synthetic Construct
GTQDETGNFDWPVSEDLNKT